(19) United States
(12) Patent Application Publication
(10) Pub. No.: US 2019/0046521 A1
(43) Pub. Date: Feb. 14, 2019

(54) Abuse-Resistant Drug Formulations

(71) Applicant: MEC Device Pharma International LLC, Fort Lauderdale, FL (US)

(72) Inventors: Hossein Omidian, Weston, FL (US); Yogesh N. Joshi, Tamarac, FL (US); Rand Husni Mahmoud Ahmad, Davie, FL (US)

(21) Appl. No.: 16/122,360

(22) Filed: Sep. 5, 2018

Related U.S. Application Data
(63) Continuation of application No. PCT/US2018/046237, filed on Aug. 10, 2018.
(60) Provisional application No. 62/543,603, filed on Aug. 10, 2017.

(51) Int. Cl. A61K 31/485; A61K 47/32; A61K 47/58

(52) U.S. Cl. CPC A61K 31/485; A61K 47/58; A61K 47/32

(57) ABSTRACT

Abuse-resistant therapeutic pharmaceutical formulations include a cationic drug and at least one anionic polymer. Particular arrangements of the cationic drug and the at least one anionic polymer prevent the cationic drug from being extracted from the formulations in solvents and conditions commonly used by drug abusers attempting to isolate the cationic drug from its formulation.

### CROSS-REFERENCE TO RELATED APPLICATIONS

The present application is a by-pass continuation under 35 U.S.C. 111(a) of international patent application number PCT/US2018/046237 filed on Aug. 10, 2018, which claims the priority of U.S. provisional patent application Ser. No. 62/543,603 filed on Aug. 10, 2017.

### STATEMENT AS TO FEDERALLY SPONSORED RESEARCH

Not applicable.

### FIELD OF THE INVENTION

The invention relates generally to the fields of pharmaceuticals. More particularly, the invention relates to drug formulations that deter abuse.

### BACKGROUND

Prescription drug abuse is an epidemic that has driven public and private efforts to combat the crisis. In 2014, more than 14,000 Americans overdosed prescription pain killers. In 2015, around 15,000 people died from prescription opioids' overdosing. The intravenous route is considered the most dangerous way of drug abuse; the drug is injected directly to the blood stream, resulting in high potential of drug overdose and subsequent health complications that may lead to death. The problem of IV drug abuse extends to include adverse health consequences such as hepatitis C and HIV infections. Moreover, prescription drug abuse results in deadly crashes as was reported in a 2010 nationwide study, which showed that 47% of the drivers involved in such crashes used a prescription drug. Associated with the negative health consequences of prescription drug abuse is the economic burden. A CDC prevention status report in 2013 revealed that illegitimate use of prescription opioid drugs resulted in annual healthcare expenditures of 72.5 billion. In 2016, the White House proposed $1.1 billion to help treat every American with an opioid-use disorder.

The FDA along with pharmaceutical industry work hand in hand to develop and evaluate abuse-deterrent formulations (ADFs). The fact that nearly half of young people who inject heroin reported abusing prescription opioids before starting to use heroin further signals the importance of such formulations. Two guidelines have been issued by the FDA, one is relevant to the evaluation and labeling of branded abuse-deterrent opioid products, and the other is relevant to the generic solid oral products. Four categories of evaluation are described in the branded products' guideline; in vitro laboratory manipulation and extraction studies, pharmacokinetic studies, clinical abuse potential studies, and post-marketing studies. The generic solid oral abuse-deterrent products' guideline specifies different tier approaches relevant to different routes of drug abuse that should be followed by manufacturers when comparing their generic version to the innovator's reference listed product. In conclusion, strict criteria are requested by the FDA for the formulation to be labelled as an ADF.

The pharmaceutical industry has developed ADFs based on different approaches. Some of these ADFs could fulfill the FDA criteria and accordingly were labeled as such, while others, despite exhibiting abuse-deterrent features have failed to acquire such labeling by not meeting the strict FDA conditions. As listed in “Abuse-Deterrent Opioids—Evaluation and Labeling” guidance, the approaches that can be employed in developing ADFs are physical/chemical barriers, agonist/antagonist combinations, aversion, delivery system, new molecular entities and prodrugs, combinations that include two or more of the mentioned approaches, and any other novel approach or technology. Most of the ADFs or products exhibiting abuse-deterrent features that are currently in the market employ the physical/chemical barriers utilizing poly(ethylene oxide) (PEO), rendering the drug products crush resistant and extraction resistant respectively and therefore claimed to impede their IV and intranasal abuse. The latest two FDA approved ADFs, Xtampza® and Vantrela™, belong to another category, i.e., the drug delivery system category. In this category, the product maintains its sustained drug release pattern, even after chewing, crushing, and/or dissolving the drug product due to the inclusion of highly hydrophobic fatty excipients in the matrix.

Despite the abuse deterrence features of the ADF products, abusers have found ways to defeat them by manipulating the environmental conditions such as temperature, shear rate, and solvents. For instance, crush resistance properties of the ADFs can generally be defeated by conducting cryogenic grinding or simply by peeling the tablet instead of crushing it. Extraction resistance by forming viscous gel of the drug product in aqueous solutions can be defeated by heating up the drug solution, addition of salts, addition of alcohol, or applying shear force and subsequent shear thinning effect. On the other hand, the sustained drug delivery system of some ADFs incorporating highly hydrophobic materials can be conquered by a multistep drug extraction.

### SUMMARY

Described herein is the development of new abuse-resistant therapeutic pharmaceutical formulations that are very effective in deterring methods of abuse. These new formulations include a cationic drug and at least one (e.g., 1, 2, 3, 4, 5 or more) anionic polymer. In some formulations, the cationic drug is physically blended with but not ionically bound to the at least one anionic polymer. In other formulations, more than 50% (e.g., 60, 70, 80, 90, 91, 92, 93, 94, 95, 96, 97, 98, or 99%) by weight of the cationic drug is ionically bound (complexed) to the at least one anionic polymer. The formulations are arranged to prevent the cationic drug from being extracted from the formulation in solvents and conditions commonly used by abusers attempting to extract drugs from drug formulations (e.g., water, hydroalcohol solutions, pH 3 solutions, acetic acid solutions, and saline at solution temperatures of 20-90° C.), while at the same time allowing drug release at the low pH sites (stomach) in the gastroinstestinal (GI) tract (e.g., in 0.1N HCl) without significant amounts (more than 10, 20, 30, or 40% by weight) of the drug re-binding the at least one anionic polymer in the less acidic locations of the GI tract (e.g., the small intestine). In this manner, the therapeutic pharmaceutical formulations described herein, prevent extraction of active drug by typical efforts employed by abusers, while at the same time allowing the intended use of the formulations (e.g., oral administration) to deliver active drug to the patient without meaningful interference from the at least one anionic polymer. In addition to binding the drug, the at least one polymer can also cause the formulation to swell or form a high viscosity gel upon exposure to a wide range of aqueous solvents. This gel effectively clogs filters and cannot be taken up in syringe needles.

Accordingly, in one aspect, the invention features an abuse-resistant therapeutic pharmaceutical formulation that includes a cationic drug and at least one anionic polymer; wherein (i) the cationic drug is physically blended with but not ionically bound to the at least one anionic polymer, or (ii) more than 50% of the cationic drug is ionically bound (complexed) to the at least one anionic polymer. The cationic drug and the at least one anionic polymer are arranged, as described below, to prevent the cationic drug from being extracted from the formulation in a solvent selected from the group consisting of water, hydroalcohol solutions, pH 3 solutions, acetic acid solutions, and saline at solution temperatures of 20-90° C.

In certain embodiments, the at least one anionic polymer includes a carboxy(methy)lated polymer or salt thereof. The carboxy(methy)lated polymer can be a carboxy(methy)lated polysaccharide (e.g., carboxymethyl cellulose) or a carboxymethyl starch. In other embodiments, the at least one anionic polymer includes a poly(acrylic acid) homopolymer, copolymer, terpolymer, or interpolymer. The poly(acrylic acid) homopolymer, copolymer, terpolymer, or interpolymer can be one crosslinked with an allyl ether of pentaerythritol, an allyl ether of sucrose, or an allyl ether of propylene as well as vinyl crosslinkers known in the art. In yet other embodiments, the at least one anionic polymer can include an anionic gum such as a xanthum gum.

In some embodiments, the cationic drug is physically blended with but not ionically bound to the poly(acrylic acid) homopolymer, copolymer, terpolymer, or interpolymer, and the formulation further includes an alkalinizing agent, e.g., wherein the weight ratio of the alkalizing agent to the poly(acrylic acid) homopolymer, copolymer, terpolymer, or interpolymer is between about 1:5 and 3:5 (preferably about 1:5 and 2:5, and more preferably about 1:5). The alkalinizing agent can be a bicarbonate salt such as sodium bicarbonate.

In some embodiments, the cationic drug is ionically bound (complexed) to the at least one anionic polymer. In such embodiments, the at least one anionic polymer can be or include a carboxy(methy)lated polymer (e.g., a carboxy(methy)lated polysaccharide such as carboxymethyl cellulose) or salt thereof, and/or a poly(acrylic acid) homopolymer, copolymer, terpolymer, or interpolymer (e.g., one which is crosslinked with an allyl ether of pentaerythritol, an allyl ether of sucrose, or an allyl ether of propylene, or vinyl crosslinkers). The cationic drug-poly(acrylic acid) homopolymer, copolymer, terpolymer, or interpolymer complex can be one made by reacting the cationic drug and the poly(acrylic acid) homopolymer, copolymer, terpolymer, or interpolymer in an aqueous solution at a pH range of greater than the pKa−1 of the poly(acrylic acid) homopolymer, copolymer, terpolymer, or interpolymer and lower than the pKa+1 of the cationic drug. The aqueous solution can include an alkalinizing agent (e.g., a bicarbonate salt such as sodium bicarbonate) which causes the pH range of the aqueous solution to be greater than the pKa−1 of the poly(acrylic acid) homopolymer, copolymer, terpolymer, or interpolymer and lower than the pKa+1 of the cationic drug such as to promote complexation of the cationic drug to the poly(acrylic acid) polymer by de-protonating the poly(acrylic acid) polymer.

The pharmaceutical formulations described herein might also include at least one aversive deterrent agent (e.g., a medicinal charcoal and/or a bentonite clay) which impart aversive properties and, in some cases, also entraps the drug. For example, a medicinal charcoal entraps the drug via adsorption and is aversive due to its powdery black nature, and a bentonite clay entraps the drug via complexation and is aversive as it is nasal irritant. Both can preferably be used at <20% (e.g., <20, 15, 10, 5, 4, 3, 2, or 1%) of the total weight of the at least one anionic polymer used in the formulation.

The formulations described herein can further include at least one non-ionic amphiphilic polymer in an amount that further prevents the cationic drug from being extracted from the formulation in a high temperature solvent or solutions selected from the group consisting of water, hydroalcohol solutions, pH 3 solutions, acetic acid solutions, and saline. The at least one non-ionic amphiphilic polymer can, e.g., be 40-80% by weight of the pharmaceutical formulation. The at least one non-ionic amphiphilic polymer can be methylcellulose.

The formulations described herein can further include a low-melting water-soluble polymer (such as poly(ethylene oxide)) or a low glass-transition temperature water-insoluble polymer such as a poly(vinyl acetate) polymer, copolymer and its blends.

Unless otherwise defined, all technical terms used herein have the same meaning as commonly understood by one of ordinary skill in the art to which this invention belongs. Commonly understood definitions of pharmaceutical terms can be found in Dictionary of Pharmacy (Pharmaceutical Heritage) 1st Edition, The Hayworth Press, 2004; and Dictionary of Pharmaceutical Medicine, Springer, 2017.

As used herein, when referring to a particular numerical value, as the context allows, the term “about” means+/−20% of that value. For example, “about 10” means 8 to 12. As another example, “about 50%” means 40% to 60%.

As used herein, when referring to a mixture of two more components, the term “physically blended” means the components are thoroughly mixed together in a formulation or subcomponent thereof, but not covalently or ionically bonded to one another.

As used herein, when referring to a mixture of two more components, the term “complexed” means the components are ionically bonded to one another before being used in the preparation of the dosage form.

Although methods and materials similar or equivalent to those described herein can be used in the practice or testing of the present invention, suitable methods and material are described below. All publications, patents, and patent applications mentioned herein are incorporated by reference in their entirety. In the case of conflict, the present specification, including definitions will control. In addition, the particular embodiments discussed below are illustrative only and not intended to be limiting.

### DESCRIPTION OF THE DRAWINGS

FIG. 1 is a graph comparing the binding efficiency of the physical blend and the complex with washed EXPLOTAB® CLV.

FIG. 2 is a graph comparing the binding efficiency of the physical blend and the complex with AC-DI-SOL®.

FIG. 3 is a series of graphs comparing the viscosities of PEO, CMC, and MC solutions (0.5-5 w/v %) at 25, 50, and 90° C.

FIG. 4 is a set of graphs comparing the viscosities of PEO, CMC, and MC solutions (2.5 and 5 w/v %) at 25, 50, and 90° C.

FIG. 5 is a set of graphs showing the gel strengths at room temperature of PEO, CMC, and MC solutions at a probe distance of 5 and 10 mm.

FIG. 6 is a set of graphs showing the gel strengths at 90° C. of PEO, CMC, and MC solutions at a probe distance of 5 and 10 mm.

FIG. 7 is a graph showing the results of a stress history analysis on 1% w/v solutions of PEO, CMC, and MC.

FIG. 8 is a series of photographs showing the gel behavior of different formulations in different extraction media.

FIG. 9 is a series of photographs showing the filtration behavior of different formulations.

FIG. 10 is a graph showing the drug release profiles of various formulations.

FIG. 11 is a series of photographs showing the gel forming behavior of different formulations in different solvents.

FIG. 12 is a graph showing the gel strength of the formulation B4 in saline at different time intervals.

FIG. 13 is a graph showing the dissolution profiles of three different B4 formulations.

FIG. 14 is a graph showing the dissolution profiles of heat-treated B4 formulations containing higher (B4P) and lower (B4P2) amounts of PEO.

FIG. 15 is a series of photographs showing the gel forming behavior of B4 and PEO formulations in different extracting solutions over time.

FIG. 16 is a series of graphs showing the equilibrium (ultimate) gel strengths of a 5 wt % PEO solution and new formulations at a probe distance of 5 and 10 mm.

FIG. 17 is a graph showing the viscosity of xanthan gum and PEO in different extracting solutions.

FIG. 18 is a graph showing the drug binding ability of xanthan gum in hydroalcoholic solutions.

FIG. 19 is a series of photographs showing the gel forming behavior of xanthan gum in different extracting solvents.

FIG. 20 is a graph showing the drug release profiles of two different xanthum gum formulations.

FIG. 21 is a graph showing the crush resistance of a xanthum gum formulation (non-treated versus heat-treated).

FIG. 22 is a graph showing the extraction stability of a therapeutic polymer-drug complex.

FIG. 23 is a graph showing the gel strength stability of the ADFs containing an in-situ gelling polymer.

FIG. 24 is a graph showing the drug release stability of the ADFs containing an in-situ gelling polymer.

FIG. 25 is a set of graphs showing the gel strength (top) and drug release (bottom) stability of a first ADF formulation containing xanthan gum.

FIG. 26 is a set of graphs showing the gel strength (top) and drug release (bottom) stability of a second ADF formulation containing xanthan gum.

FIG. 27 is a table showing the effect of amount of sodium bicarbonate on Carbomer binding to drug.

FIG. 28 is a graph showing the binding efficiency of Carbomer to drug in a physical blend formulation in various solvents.

FIG. 29 is a graph showing the binding efficiency of Carbomer-drug complexes in various solvents.

FIG. 30 is a graph showing the drug release profiles of two different Carbomer-drug complexes in 0.1N HCl.

FIG. 31 is a set of graphs showing the re-binding of a high-loaded Carbomer-drug complex in water and phosphate buffer.

FIG. 32 is a graph comparing drug-binding efficiencies of drug-Carbomer physical blends and drug-Carbomer complexes.

### DETAILED DESCRIPTION

Described herein are abuse-resistant therapeutic pharmaceutical formulations that include a cationic drug and at least one anionic polymer. Particular arrangements of the cationic drug and at least one anionic polymer prevent the cationic drug from being extracted from the formulations in solvents and conditions commonly used by drug abusers attempting to isolate a cationic drug from its formulation.

### General Methods

Methods involving conventional pharmaceutical formulation are described in Pharmaceutical Formulation: The Science and Technology of Dosage Forms (Drug Discovery) (1st Edition), Royal Society of Chemistry (2018); Handbook of Pharmaceutical Manufacturing Formulations, Vol. 1: Compressed Solid Products (2nd Edition), Informa Healthcare USA (2009); and Remington's Pharmaceutical Sciences (20th Edition), Williams and Wilkins (2000). Conventional techniques in polymer chemistry are described in Polymer Science and Technology (3rd Edition), Pearson Education (2014).

### Abuse-Resistant Therapeutic Pharmaceutical Formulations

Abuse-resistant therapeutic pharmaceutical formulations include a cationic drug and at least one (e.g., 1, 2, 3, or more) anionic polymer. In some formulations, the cationic drug can be physically blended with but not ionically bound to the at least one anionic polymer. In other formulations, more than 50% (e.g., 60, 70, 80, 90, 91, 92, 93, 94, 95, 96, 97, 98, or 99%) by weight of the cationic drug can be ionically bound (complexed) to the at least one anionic polymer. The formulations are arranged to prevent the cationic drug from being extracted from the formulation in solvents and conditions commonly used by abusers attempting to extract drugs from drug formulations (e.g., water, hydroalcohol solutions, pH 3 solutions, acetic acid solutions, and saline at solution temperatures of 20-90° C.), while at the same time allowing drug release at the low pH environment in the gastroinstestinal (GI) tract (e.g., in 0.1N HCl) without significant amounts (more than 10, 20, 30, or 40% by weight) of the drug re-binding the at least one anionic polymer in the less acidic locations of the GI tract (e.g., the small intestine). In this manner, the therapeutic pharmaceutical formulations described herein, prevent extraction of active drug by typical efforts employed by abusers, while at the same time allowing the intended use of the formulations (e.g., oral administration) to deliver the active drug to the patient without meaningful interference from the at least one anionic polymer.

The anionic polymer and its association with the cationic drug is selected such that in solvents and conditions commonly used by abusers attempting to extract drugs from drug formulations, at least 15% (but preferably at least 20, 30, 40, 50, 60, 70, 80, 90, 91, 92, 93, 94, 95, 96, 97, 98, or 99%) by weight of the cationic drug is retained bound to the at least one anionic polymer. In preferred embodiments, the anionic polymer and its association with the cationic drug is selected such that at temperatures between 20-90° C., in water, at least 70% (but preferably at least 80, 90, 91, 92, 93, 94, 95, 96, 97, 98, or 99%) by weight of the cationic drug is retained bound to the at least one anionic polymer. In preferred embodiments, the anionic polymer and its association with the cationic drug is selected such that at temperatures between 20-90° C., in pH3 solutions, at least 70% (but preferably at least 80, 90, 91, 92, 93, 94, 95, 96, 97, 98, or 99%) by weight of the cationic drug is retained bound to the at least one anionic polymer. In preferred embodiments, the anionic polymer and its association with the cationic drug is selected such that at temperatures between 20-90° C., in 40% ethanol, at least 40% (but preferably at least 60, 70, 80, 90, 91, 92, 93, 94, 95, 96, 97, 98, or 99%) by weight of the cationic drug is retained bound to the at least one anionic polymer. In preferred embodiments, the anionic polymer and its association with the cationic drug is selected such that at temperatures between 20-90° C., in saline (0.9% by weight NaCl) at least 20% (but preferably at least 30, 40, 50, 60, 70, 80, 90, 91, 92, 93, 94, 95, 96, 97, 98, or 99%) by weight of the cationic drug is retained bound to the at least one anionic polymer. In preferred embodiments, the anionic polymer and its association with the cationic drug is selected such that at temperatures between 20-90° C., in 0.1M acetic acid, at least 50% (but preferably at least 60, 70, 80, 90, 91, 92, 93, 94, 95, 96, 97, 98, or 99%) by weight of the cationic drug is retained bound to the at least one anionic polymer. And in particularly preferred embodiments, the anionic polymer and its association with the cationic drug is selected such that at temperatures between 20-90° C., in water, pH3 solutions, 40% ethanol, saline, and 0.1 M acetic acid is at least 70% by weight of the cationic drug is retained bound to the at least one anionic polymer.

### Cationic Drugs

Any cationic drug (weak base or salt of a weak base) suitable for use in the technologies described herein might be used. Prescription drugs that are often the subject of abuse (e.g., those that cause physical or psychological dependence or use disorder) are preferred. Examples of these include opioids and morphine derivatives, depressants, stimulants, and others (such as dextromethorphan). Examples of opioids and morphine derivatives include codeine, morphine, methadone, tramadol, fentanyl and analogs thereof, oxycodone, hydrocodone, hydromorphone, oxymorphone, meperidine, buprenorphine, and propoxyphene. Examples of depressants include barbiturates, benzodiazepines, and sleep medications such as zolpidem, zaleplon, and eszopiclone. Examples of stimulants include amphetamines and methylphenidate. Other cationic drugs intended for other therapeutic applications may include epinephrine (and its salts) and antagonists such as naloxene and naltroxene (and their salts).

### Anionic Polymers

The at least one anionic polymer used in the formulations described here can be a single anionic polymer or a blend of 2 or more (e.g., 3, 4, or 5) different anionic polymers. Any anionic polymer suitable for use in the technologies described herein might be used. A suitable anionic polymer for many formulations is a carboxy(methy)lated polymer or salt thereof. The carboxy(methy)lated polymer can be a carboxy(methy)lated polysaccharide (e.g., carboxymethyl cellulose such as croscarmellose sodium (e.g., AC-DI-SOL®)) or a carboxymethyl starch possessing different degrees of substitutions (functionality) and crosslinking (such as EXPLOTAB® regular grade, EXPLOTAB® low pH, EXPLOTAB® CLV, GLYCOLYS®, GLYCOLYS® LV, VIVASTAR® PSF, and GLYCOLYS® LM).

Another suitable anionic polymer for many formulations is a poly(acrylic acid) homopolymer, copolymer, terpolymer, or interpolymer. The poly(acrylic acid) homopolymer, copolymer, terpolymer, or interpolymer can be one crosslinked with an allyl ether of pentaerythritol, an allyl ether of sucrose, or an allyl ether of propylene e.g., CARBOPOL® (all grades including 71G NF, 971P NF, 974P NF, 980 NF, 981 NF, 5984 EP, ETD 2020 NF, Ultrez 10 NF, 934 NF, 934P NF, 940 NF, 941 NF, and 1342 NF), and those crosslinked using vinyl crosslinkers. Yet another suitable anionic polymer for many formulations is an anionic gum such as a xanthum gum. For formulations where the cationic drug is ionically bound (complexed) to the at least one anionic polymer, the at least one anionic polymer can be or include a carboxy(methy)lated polymer (e.g., a carboxy(methy)lated polysaccharide such as carboxymethyl cellulose) or salt thereof, and/or a poly(acrylic acid) homopolymer, copolymer, terpolymer, or interpolymer (e.g., one which is crosslinked with an allyl ether of pentaerythritol, an allyl ether of sucrose, or an allyl ether of propylene, or vinyl crosslinkers).

### Physical Blends

In those formulations where the cationic drug is physically blended with but not ionically bound to the at least one anionic polymer, the weight ratio of cationic drug to at least one anionic polymer in the formulation should be selected to ensure that at least 40% (e.g., at least 40, 50, 60, 70, 80, 90, 95, of 99%) by weight of the cationic drug in the formulation becomes bound to the at least one anionic polymer upon exposure to solvents and conditions commonly used by abusers attempting to extract drugs from drug formulations (e.g., water, hydroalcohol solutions, pH 3 solutions, acetic acid solutions, and saline, at solution temperatures of 20-90° C.). Depending on the particular at least one anionic polymer used in the formulation, the weight ratio of cationic drug to at least one anionic polymer in the formulation can be about 1:1, 1:2, 1:3, 1:4, 1:5, 1:10, or 1:20. For example, where carboxymethyl cellulose, carboxymethyl starch, or poly(acrylic acid) is the at least one anionic polymer, the weight ratio of cationic drug to at least one anionic polymer is preferably in the range of 1:4 to 1:16 (e.g., 1:6 to 1:10, or about 1:8). In those formulations where the cationic drug is physically mixed or blended with the poly(acrylic acid) polymers, copolymers and interpolymers, a solid alkalizing agent (preferably a bicarbonate) is also included in the blend to facilitate the ionization of the poly(acrylic acid) polymer and its complexation with the cationic drug in different extracting solutions. The weight ratio of the alkalizing agent to poly(acrylic acid) polymer, copolymer or interpolymer is preferably 1:5. Based on the methods and results described in the Examples section below, optimal cationic drug to at least one anionic polymer weight ratios to be used in making any particular formulation can be determined empirically, e.g., by trying different ratios in the extraction experiments described in the Examples section. These ratios might also be adjusted to enhance the ability of a formulation to form gels in solvents, to resist stress, to resist filterability, and to resist syringeability.

### Complexes

In those formulations where more than 50% by weight of the cationic is ionically bound (complexed) to the at least one anionic polymer, the weight ratio of cationic drug to at least one anionic polymer in the complexation reaction should be selected in accordance with the amount of drug loading onto the at least one anionic polymer that is desired. Depending on the particular at least one anionic polymer used in the formulation and the amount of drug loading desired, the weight ratio of cationic drug to at least one anionic polymer in the formulation can be about 3:1, 2:1, 1:1, 1:2, 1:3, 1:4, 1:5, 1:10, or 1:20. Generally, more a higher weight ratio of cationic drug to at least one anionic polymer is used to achieve higher drug loading onto the polymer. For example, where carboxymethyl cellulose, carboxymethyl starch, or poly(acrylic acid) is the at least one anionic polymer, the weight ratio of cationic drug to at least one anionic polymer is preferably in the range of 2:1 to 1:15 (e.g., 1:1 to 1:10, or about 1:1.3 or 1:8). Based on the methods and results described in the Examples section below, optimal cationic drug to at least one anionic polymer weight ratios to be used in any particular formulation can be determined empirically, e.g., by trying different ratios in the extraction experiments described in the Examples section. These ratios might also be adjusted to enhance the ability of a formulation to form gels in solvents, to resist filterability, and to resist syringeability.

To enhance complexation of the cationic drug to some anionic polymers (e.g., poly(acrylic acid) polymers), the complexation reaction can be carried out in an aqueous solution at a pH range of greater than the pKa−1 (preferably greater than the pKa) of the anionic polymer and lower than the pKa+1 (preferably lower than the pKa) of the cationic drug such as to promote complexation of the cationic drug to the anionic polymer by de-protonating the poly(acrylic acid) polymer. An alkalinizing agent (e.g., a bicarbonate salt such as sodium bicarbonate, or a carbonate, a phosphate, hydroxide of sodium or potassium, magnesium carbonate, magnesium hydroxide, ammonium carbonate, ammonium bicarbonate, magnesium oxide, calcium hydroxide, alkanol amines, amino sugars, citrates, acetates, or mixtures thereof that causes the pH range of the aqueous solution to be greater than the pKa−1 of the poly(acrylic acid) homopolymer, copolymer, terpolymer, or interpolymer and lower than the pKa+1 of the cationic drug may be added to the reaction mixture (e.g., at a weight ratio of the alkalizing agent to polymer or between about 1:5 and 3:5, 1:5 and 2:5, or 1:5) this purpose.

### Optimizing Abuse Resistance

Optimal formulations of cationic drug and at least one anionic polymer for any given desired abuse resistance characteristics can be prepared by the methods described herein by varying the reagents and their amounts used in the formulations or the complexation reactions. The abuse resistance characteristics of any particular formulation can be determined using the assays described in the Examples section as well as others known in the art. For example, resistance of a particular formulation to extraction in solvents and conditions commonly used by abusers attempting to extract drugs from drug formulations (e.g., water, hydroalcohol solutions, pH 3 solutions, acetic acid solutions, and saline, at solution temperatures of 20-90° C.) can be determined by thoroughly mixing the particular formulation in each solvent (typically by vortexing for 30 seconds), centrifuging the mixture to allow for the separation of the free drug in the supernatant from the polymer-bound drug in the sediment, appropriately diluting the supernatant (typically 1 to 10) in the solvent, filtering the diluted supernatant (e.g., through a 0.2 μm syringe filter), and subjecting the filtrate to UV spectrophotometry to determine the drug concentration therein. The percentage of the drug extracted in solution can be calculated relevant to established calibration curves in each solvent, and the percentage of drug binding can be determined from the mass balance.

To examine the ability of a particular formulation to release drug in the GI tract, the formulation can be added to 0.1 N HCl at 37±0.5° C. with mixing for different time periods (e.g., 15, 30, and 60 min) to mimic stomach conditions. At each of the time periods samples are withdrawn and analyzed for free drug by the method described above. Because polymer re-binding (and inactivation) in post-stomach digestive tract sites (particularly the small intestine) can be a concern, following the above assay can be followed a second assay performed in water and pH 7.5 phosphate buffer to simulate conditions in the intestinal media as described in more detail in Example 1.

Other methods for assessing the abuse resistance characteristics of particular formulations such as crushability/particle size assays, gel formation/viscosity assays, stress resistance, filterability, and syringeability can be performed as described in the Examples section.

### Other Components

The pharmaceutical formulations described herein might also include other components that contribute to abuse resistance. For example, pharmaceutical formulations described herein might include at least one non-ionic amphiphilic polymer (e.g., methylcellulose) that can further minimize drug extraction over a wide temperature range, increase viscosity of the formulation when exposed to a solvent, improve stress history (the ease at which the solution can be drawn up into the syringe after it undergoes multiple aspirations, pulling in and out of syringe), and/or increase the force required to aspirate the solution into syringe (decrease syringeability). The combination of a non-ionic amphiphilic polymer with another polymer such as carboxymethylcellulose can further enhance abuse resistance. In preferred embodiments, the at least one non-ionic amphiphilic polymer can make up about 40-80% of the weight of the pharmaceutical formulation. The pharmaceutical formulations described here can further include poly(ethylene oxide) or poly(vinyl acetate) polymers, copolymers, or blends.

Still other agents that contribute abuse resistance can be included in the pharmaceutical formulations described herein. For instance, at least one aversive deterrent agent (e.g., a medicinal charcoal and/or a bentonite clay) which imparts aversive properties and, in some cases, also entraps the drug can be included. Examples of aversive deterrent agents include medicinal charcoals which can entrap the drug via adsorption and provide abuse aversion due to their powdery black nature, and bentonite clays which entrap drugs via complexation and provide abuse aversion as a nasal irritant. Both can preferably be used at <20% (e.g., less than 20, 15, 10, 5, 4, 3, 2, or 1%) of the total weight of the at least one anionic polymer used in the formulation.

The pharmaceutical formulations described herein can also include other inactive excipients needed to prepare the final dosage forms. Such inactive excipients provide neither therapeutic nor abuse deterrent properties.

### Formulation Forms

Suitable forms of the pharmaceutical formulations described herein include but are not limited to powders, granules, suspensions, emulsions, and gels that can be manufactured in the final form of tablets, capsules, patches, films, suppositories, and liquid dosage forms. The forms can be any shape, including regular or irregular shape depending upon the needs of the artisan. Compressed tablets including the pharmaceutical formulations described herein can be direct compression tablets or non-direct compression tablets. Some of these forms can be made by wet granulation, and dry granulation (e.g., slugging or roller compaction). Additionally, the pharmaceutical tablets described herein can undergo a thermal treatment either before, concurrent, or after tabletting.

The anionic polymer of this invention can also be tailor-made to further improve abuse-deterrent properties in majority of solvents used for drug extraction. The acrylic acid monomer can be neutralized, copolymerized or terpolymerized with more than one monomer, each providing maximum deterrence in a specific solvent. For instance, a polymer synthesized from acrylic acid, potassium acrylate, acrylamide, and potassium sulfopropyl acrylate (or alternatively poly(2-acrylamido 2-methyl-propane sulfonic acid (AMPS)) can improve deterrence in water (primarily due to acrylic and sulfopropyl acrylate), hydroalcoholic solutions (primarily due to acrylamide), and saline solutions (primarily due to sulfopropyl acrylate and AMPS). Alternatively, the corresponding homopolymers can be blended to achieve similar deterrence.

### EXAMPLES

### Example 1: Minimizing Free Drug Available for Extraction Using Crosslinked Starch and Cellulose

The binding efficiency of two anionic deterrents (carboxymethyl starch and carboxymethyl cellulose) to cationic opioid drug (Dextromethorphan HBr) in different aqueous solvents most commonly used by abusers for IV administration was evaluated. The binding efficiency study was conducted in two forms; the physical blend and the complexation forms. In the physical blend study, the anionic excipient was physically mixed with the free drug in the formulation, while in the complexation study, the excipient and the drug were complexed together in a previous step and the formed powder complex was incorporated in the formulation. Drug release from the formulations under regular therapeutic use was evaluated in 0.1 N HCl dissolution medium, followed by examining re-binding potential under intestinal environment, represented by Stage II dissolution study using water and phosphate buffer (pH 7.5) as dissolution media. The binding rebinding studies in SGF followed by SIF or phosphate buffer (pH 7.5) is, in particular, necessary for extended release ADFs where the release of the drug will occur over long period of time (e.g., 24 hours) across small and large intestines.

Three grades of crosslinked carboxymethyl starch (EXPLOTAB® regular grade, EXPLOTAB® low pH, and EXPLOTAB® CLV) were donated from Mutchler Inc. (JRS Pharma, Germany). Crosslinked carboxymethyl cellulose (AC-DI-SOU)) was purchased from FMC Biopolymer (Belgium). The model drug DEX HBr USP was purchased from Letco Medical (USA). The chemical reagents used in preparing aqueous solutions included hydrochloric acid fuming 37% Ph. Eur. (Merck KGaA, Germany), sodium chloride (Merck KGaA, Germany), Reagent alcohol 100% (Decon Laboratories Inc., USA), monobasic potassium phosphate (J.T. Baker, USA), sodium hydroxide (J.T. Baker, Sweden), acetic acid glacial (J.T. Baker, USA), and distilled water.

The prepared aqueous solutions included 0.1 N hydrochloric acid (8.3 mL of HCl 37% up to 1 L in distilled water), pH 3 solution (distilled water, adjusted to pH 3 by HCl), normal saline (9 g sodium chloride dissolved in 1 L solution), ethanol 40% v/v (40 mL pure alcohol mixed with 60 mL distilled water), pH 7.5 phosphate buffer (40.8 g of monobasic potassium phosphate and 9.6 g of sodium hydroxide in 6 L distilled water, adjusted to pH of 7.5 with phosphoric acid or 2 N sodium hydroxide [16]), 0.83 M acetic acid (23.73 mL of glacial acetic acid up to 500 mL in distilled water), 0.5 M acetic acid (14.30 mL of glacial acetic acid up to 500 mL in distilled water), and 0.1 M acetic acid (2.86 mL of glacial acetic acid up to 500 mL in distilled water).

To prepare a drug polymer physical blend, in a scintillation vial, the free cationic opioid drug (DEX HBr) and the anionic deterring agent (either carboxymethyl starch or carboxymethyl cellulose) were weighed and physically mixed in triplicates in a weight ratio of 1:8 respectively to mimic a crushed tablet intended for IV abuse. Different aqueous solvents (10 mL) most commonly used by abusers were added to the physical blend in the vial as an extraction medium. The solvents included water, pH 3 solution, normal saline, 40% v/v ethanol, 0.83 M acetic acid, 0.5 M acetic acid, and 0.1 M acetic acid. The mixture was vortexed (Fisher Scientific) for 30 seconds, and centrifuged (Beckman Coulter, Allegra™ X-22R) at 1500 rpm for 5 minutes to allow for the separation of the free drug in the supernatant from the polymer bound drug in the sediment. The supernatant solution was diluted (1.0 ml into 10.0 ml solution) in each solvent and then filtered by passing through 0.2 μm syringe filter. Drug concentration was determined by UV spectrophotometer (Shimadzu UV-1700 Spectrophotometer) at 276 nm. The percentage of the drug extracted in solution was calculated relevant to established calibration curves in each solvent, and the percentage of drug binding was determined from the mass balance.

Different grades of carboxymethyl starch (EXPLOTAB® regular grade, EXPLOTAB® low pH, and EXPLOTAB® CLV) were tested for their binding efficiency and compared to each other. Moreover, EXPLOTAB® CLV was washed with water and tested for its binding efficiency after the washing step and compared to the non-washed polymer. The other anionic polymer, carboxymethyl cellulose (AC-DI-SOL®) was tested for its binding efficiency as well.

To prepare a complex composed of the opioid drug (DEX HBr) and the abuse deterring agent (washed EXPLOTAB® CLV or AC-DI-SOL®) were mixed in 500 mL water overnight at a 1:8 drug to excipient ratio. The mixtures were left standing for 12 hours to allow for sedimentation of the undissolved excipient along with the portion of the drug bound to it. The supernatant (water and the portion of the dissolved drug) was collected and measured by UV spectrophotometer (276 nm) to determine the absorbance value of DEX HBr in the solution, which was discarded afterwards. Washing the sediment in water continued until the supernatant's absorbance value approached zero, indicating that the wash out is free of drug. Filtration under vacuum was conducted and the wet drug-polymer complex retained on the filtration base was dried in an oven at 65° C. overnight. The dried powder complex was milled by ball mill (Retsch, MM200) to particle size of 45 μm<X<125 μm. Binding efficiencies of EXPLOTAB® CLV and AC-DI-SOL® to the drug in its bound (complexed) form was determined in different extracting solvents by weighing given amounts of the prepared complexes (equivalent to 25 mg drug) into scintillation vials (Table 1). The extracting solvents (10.0 mL), including water, pH 3 solution, normal saline, 40% v/v ethanol, 0.83 M acetic acid, 0.5 M acetic acid, and 0.1 M acetic acid were added to different vials in triplicates. The mixtures were vortexed for 30 seconds and centrifuged at 1500 rpm for 5 minutes. The supernatant solution was diluted (1.0 ml into 10.0 ml solution) in each solvent, and then filtered by passing through a 0.2 μm syringe filter. The drug concentration was determined by UV spectrophotometer at 276 nm to determine the percentage of the extracted drug in solution relevant to established calibration curves in different solvents. Finally, the percentage of the drug that remained bound in the complex was determined from the mass balance. Moreover, another complex was prepared as described above at a ratio of 1:1.3 drug to polymer. The loading capacity of the 1:8 and 1:1.3 complexes (denoted as low-loaded and high-loaded complexes, respectively) was determined quantitatively by dissolution testing in 0.1 N HCl (900 mL). A qualitative confirmatory testing was conducted using an IR spectrophotometer (PerkinElmer, Spectrum 100 FT-IR Spectrometer). The binding efficiency of the high loaded complexes was determined in different extracting solvents and compared to that of the low-loaded complexes. Table (1) provides the amounts of each complex, equivalent to 25 mg of DEX HBr.

TABLE 1
The amounts of EXPLOTAB ® CLV-drug and AC-DI-SOL ®-drug complexes equivalent
to 25 mg drug.
Complex

| Low-loaded (1:8) | High-loaded (1:1.3) | Low-loaded (1:8) | High-loaded (1:1.3) |
|---|---|---|---|
| DEX HBr-EXPLOTAB ® | DEX HBr- | DEX HBr-AC-DI- | DEX HBr-AC-DI- |
| CLV | EXPLOTAB ® CLV | SOL ® | SOL ® |
| 278.8 mg | 239.3 mg | 248.9 mg | 66.5 mg |

Method to measure drug release in 0.1N HCl under therapeutic use: 500 mg tablets were prepared from the above described complexes by the direct compression method. Placebo tablets were used as control. Tables (2) and (3) summarize the compositions of the tablets.

TABLE 2
Tablets prepared from drug-EXPLOTAB ® CLV and drug-AC-DI-SOL ® complexes.

| | Complex | | | |
|---|---|---|---|---|
| | Low-loaded | High-loaded | | |
| | DEX HBr- | DEX HBr- | Low-loaded | High-loaded |
| | EXPLOTAB ® | EXPLOTAB ® | DEX HBr- | DEX HBr-AC- |
| | CLV | CLV | AC-DI-SOL ® | DI-SOL ® |
| Complex/tablet | 278.8 mg | 239.3 mg | 248.9 mg | 66.5 mg |
| AVICEL ®/tablet | 221.2 mg | 260.7 mg | 251.1 mg | 433.5 mg |

TABLE 3
Control (placebo) tablets composition.

| | Complex Placebo | | | |
|---|---|---|---|---|
| | Low-loaded | High-loaded | | |
| | DEX HBr- | DEX HBr- | Low-loaded | High-loaded |
| | EXPLOTAB ® | EXPLOTAB ® | DEX HBr- | DEX HBr- |
| | CLV | CLV | AC-DI-SOL ® | AC-DI-SOL ® |
| Deterrent/tablet | 253.8 mg | 214.3 mg | 223.9 mg | 41.5 mg |
| AVICEL ®/tablet | 246.2 mg | 285.7 mg | 276.1 mg | 458.5 mg |

The tablets underwent dissolution testing as per the USP method (U.S. Pharmacopeial Convention. General Chapters: <711> DISSOLUTION. USP 40-NF 35, 2017: p. 588-98). Dissolution apparatus (II) paddle at 50 rpm speed and 900 mL of 0.1 N HCl at 37±0.5° C. Samples (5 mL) were withdrawn from the dissolution vessels after 15 min., 30 min, and 60 min. The withdrawn amounts of the dissolution medium were immediately replaced after each sampling point using fresh 0.1 N HCl. The percentage of drug released from the different formulations was measured by a UV spectrophotometer at 276 in reference to a calibration curve established in 0.1N HCl.

Method to measure re-binding potential in water and pH 7.5 phosphate buffer under therapeutic use: upon oral administration and under the physiological conditions of the body, the drug reaches the intestine after passing by the stomach. Thus, re-binding potential between the abuse-deterrent anionic polymers and cationic drug was examined under the intestinal environment which is characterized of higher pH value in comparison with the gastric environment. Therefore, stage I dissolution study in 0.1 N HCl described above was followed by stage II dissolution in simulated intestinal media, water and pH 7.5 phosphate buffer.

The dissolution medium of stage I (0.1 N HCl), containing the released soluble drug from the tablets was dumped, while the insoluble deterring agent (EXPLOTAB® CLV or AC-DI-SOL®) was kept in each dissolution vessel. 900 mL of the new medium (either water or phosphate buffer) were added to the three placebo vessels which already contain protonated deterring agent from stage I dissolution. Drug solution (25 mg drug in 900 mL water or 25 mg drug in 900 mL phosphate buffer) was added to the other three dissolution vessels which again already contain protonated deterring agent from stage I dissolution. The test was run utilizing dissolution apparatus (II) paddle at 50 rpm speed and 900 mL of water or pH 7.5 phosphate buffer as dissolution media. Samples (5 mL) were withdrawn from the dissolution vessels after 15 min., 30 min., 1 h., 2 h., 6 h., 8 h., 12 h., and 24 h. Withdrawn solutions were immediately replaced after each sampling point by fresh dissolution medium. Drug concentration in each dissolution vessel was determined by a UV spectrophotometer at 276 nm, and the percentage of the free drug available was calculated in reference to calibration curves established in water and pH 7.5 phosphate buffer.

Drug binding under IV abuse conditions was evaluated. For the drug polymer blends, the anionic polymer (EXPLOTAB® regular grade, EXPLOTAB® low pH, EXPLOTAB® CLV, washed EXPLOTAB® CLV), and AC-DI-SOL®) was blended with the free cationic drug (DEX HBr), and 10 mL of different aqueous extracting solvents were added to the blend simulating the IV abuse conditions in terms of the most commonly used solvents by abusers and the maximum volume that can be injected as bolus. The aqueous solvent is responsible for functionalizing the deterring anionic polymer by causing its dissociation into carboxylate negative ions, which will provide binding sites to the cationic opioid drug and subsequent decrease in its free amount available for extraction and injection. The results of the study (Table 4) show a difference in the mean of % binding between the different grades of EXPLOTAB® (carboxymethyl starch) not exceeding 5% in the different extracting solvents except in 40% v/v ethanol, where the difference was more than 10% with the low pH grade. This might be attributed to the lower sodium glycolate content of this grade (2.0-3.4%) compared to the regular and CLV grades (2.8-4.2%), affecting the functionality of the polymer and its dissociation in challenging extracting media such as the less polar 40% ethanol solvent. By changing the extraction medium, different degrees of binding were achieved. Maximum binding (55-65%) was achieved in water and pH 3 solution, while lower binding (20-35%) was achieved in normal saline and 40% v/v ethanol. The binding between the anionic polymer and the cationic drug is based on electrostatic interaction between the two species, which is inversely affected by the presence of ions derived from normal saline solution. As mentioned earlier, the binding is dependent on the dissociation of the carboxyl groups into anionic binding sites, which is affected by the polarity of the solvent and therefore is expected to be less efficient in 40% v/v ethanol.

Carboxymethyl starch contains sodium chloride up to 7.0%. This ionic content interferes with the binding efficiency of the anionic polymer to the cationic opioid drug. Therefore, the binding efficiency of one of the carboxymethyl starch grades (EXPLOTAB® CLV) was examined after washing the material with water to lower its sodium chloride content. The obtained results were compared to that of the non-washed polymer. The results in Table 4 show no change in the binding level between the washed and non-washed polymer in normal saline due to the ions availability from the extracting medium itself and thus maintaining the ionic interference even after washing the excipient. Around 10% binding increase was achieved with the washed polymer in water and pH 3 solution. Interestingly, significant increase (almost twice as much) in the binding level was achieved in 40% ethanol. Washing out the sodium chloride ions had a greater impact in ethanol as it initially (before washing) showed lower binding values compared to water. The binding efficiency was also examined in different concentrations of acetic acid, a less polar solvent that provides carboxylate ions in the solution. As the concentration of the acid increased, the binding decreased presumably due to the presence of more carboxylate ions in the solution. The newly dissociated carboxylate ions can interact with the drug in a competitive manner with the deterrent agent, resulting in lower binding of the deterrent agent, and hence greater drug extraction in the presence of concentrated acetic acid solutions.

AC-DI-SOL®, a cross linked carboxymethyl cellulose with higher functionality and lower sodium chloride content (not more than 0.5%) was compared to carboxymethyl starch in the form of a physical blend under the IV abuse conditions. The results (Table 4) showed better binding efficiency in comparison with the non-washed EXPLOTAB® grades containing higher sodium chloride content, except in normal saline, where the binding was almost comparable due to the ionic effect derived from the solvent in both cases. In comparison to the washed EXPLOTAB® CLV possessing almost similar sodium chloride content as AC-DI-SOL®, the binding was almost similar in the less polar solvents (40% v/v ethanol and acetic acid solutions) due to comparable functional groups activation because of the limited dissociation in such extracting solvents. The effect of AC-DI-SOL® higher functionality was evident in polar water and pH 3 solution, whereby more than 10% increase in the binding level was achieved.

TABLE 4
Binding efficiency of different anionic polymers to cationic DEX HBr in different
aqueous extracting solvents.
% Binding ± SD

| | EXPLOTAB ® | EXPLOTAB ® | EXPLOTAB ® CLV | | |
|---|---|---|---|---|---|
| | Regular | low pH | Non- | | AC-DI- |
| | Non-washed | Non-washed | washed | Washed | SOL ® |
| Water | 57% ± 0.5 | 59% ± 0.8 | 61% ± 0.5 | 71% ± 1.2 | 86% ± 0.2 |
| pH 3 solution | 56% ± 2.9 | 56% ± 0.1 | 61% ± 2.7 | 72% ± 0.8 | 83% ± 0.3 |
| Normal saline | 29% ± 0.4 | 28% ± 0.4 | 29% ± 1.4 | 29% ± 0.4 | 20% ± 0.6 |
| 40% v/v | 34% ± 2.3 | 21% ± 0.9 | 33% ± 0.4 | 60% ± 0.3 | 57% ± 0.9 |
| ethanol | | | | | |
| Acetic acid, | | | | 29% ± 0.2 | 27% ± 1.3 |
| 0.83M | | | | | |
| Acetic acid, | | | | 37% ± 0.4 | 32% ± 1.8 |
| 0.5M | | | | | |
| Acetic acid, | | | | 56% ± 0.2 | 53% ± 2.1 |
| 0.1M | | | | | |

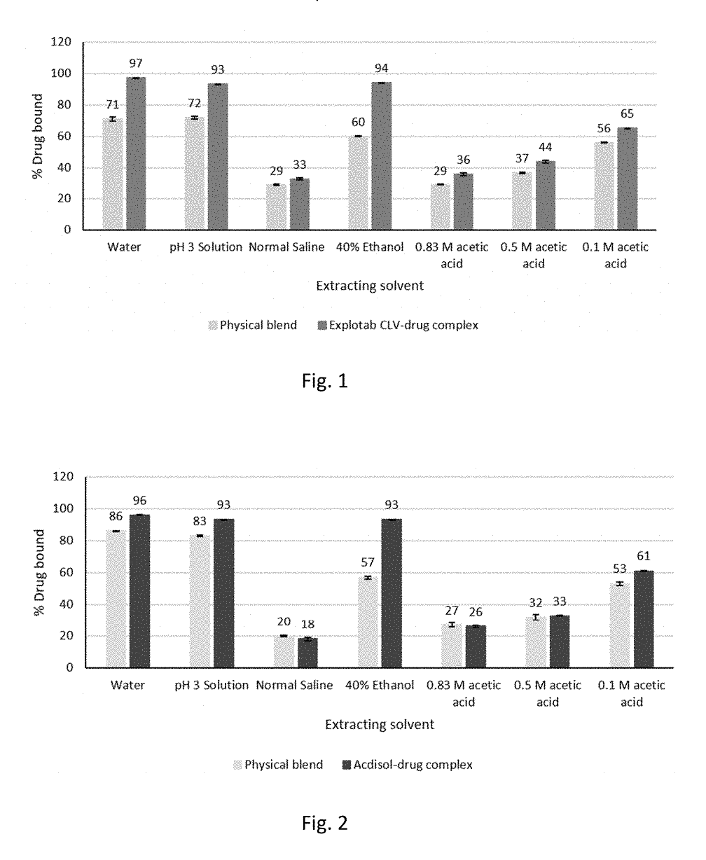

Drug Polymer Complex: a complex was formed between the cationic opioid drug (DEX HBr) and the anionic deterring polymer (washed EXPLOTAB® CLV and AC-DI-SOL®) in a ratio of 1:8. Binding efficiency of the deterring agents to the drug in its bound (complexed) form was determined in different extracting solvents by weighing given amounts of the prepared complexes (equivalent to 25 mg drug) into scintillation vials (Table 1) and the subsequent addition of 10 mL extracting solvent. The results (FIG. 1) of drug-EXPLOTAB® complex versus the physical blend showed around 20-25% increase in the binding efficiency in both water and pH 3 solution, around 35% increase in 40% v/v ethanol, and no significant difference (less than 10%) in normal saline and the acetic acid solutions. Thus, drug extraction is impeded upon drug complexation. More than 90% binding was achieved in water, pH 3 solution and 40% ethanol upon drug complexation compared to 71%, 72%, and 60% binding respectively in the physical blend studies. The more significant binding improvement achieved in ethanol (35% versus 20-25%) is justifiable by its initial lower binding level in the physical blend study, whereby the lower ethanol polarity governed the dissociation (activation) of EXPLOTAB®. But upon drug complexation, the polymer was activated to its maximum by water, before the subsequent addition of ethanol that had no effect on EXPLOTAB® dissociation afterward. The insignificant difference between the complex and physical blend results achieved in normal saline and acetic acid solutions is attributable to the ionic interference originating from the sodium chloride and carboxylate ions, respectively. The same applies to AC-DI-SOL®-DEX HBr complex (FIG. 2).

This study also included the formation of another complex of 1:1.3 drug to polymer ratio. The purpose of this study was to determine the drug loading capacity of EXPLOTAB® CLV and AC-DI-SOL®. The two complexes, 1:8 and 1:1.3 drug-polymer ratios were identified as low-loaded and high-loaded complexes, respectively. The obtained results utilizing dissolution testing in 0.1 N HCl showed almost comparable drug loading capacity with the 1:8 and 1:1.3 drug-EXPLOTAB® complexes, indicating a saturated drug complexation at the lower ratio. Drug complexation with AC-DI-SOL® showed significantly higher loading with the 1:1.3 drug-polymer ratio compared to the 1:8 ratio, pointing out to the higher functionality of AC-DI-SOL® versus EXPLOTAB® which was responsible for better loading as drug concentration increased. The drug loading capacity results derived from the dissolution testing are summarized in Table 5.

Furthermore, a qualitative analysis by IR was conducted to determine the correlation coefficient between the low- and high-loaded complexes of each polymer composition. The IR spectra confirmed the formation of drug-polymer complexes, and the correlation coefficient values confirmed the results obtained from the dissolution testing, where the coefficients were 0.98 and 0.85 for the EXPLOTAB® and AC-DI-SOL® complexes, respectively, indicating similar drug loading in the EXPLOTAB® complexes and different loading in the AC-DI-SOL® complexes.

TABLE 5
Drug loading capacity of different drug-polymer complexes.
Amount of loaded drug/1 g polymer

| | |
|---|---|
| Low-loaded DEX HBr-EXPLOTAB ® CLV | 99 mg |
| High-loaded DEX HBr-EXPLOTAB ® CLV | 117 mg |
| Low-loaded DEX HBr-AC-DI-SOL ® | 112 mg |
| High-loaded DEX HBr-AC-DI-SOL ® | 602 mg |

A drug binding efficiency study was conducted on the high-loaded complexes of EXPLOTAB® CLV and AC-DI-SOL® by weighing given amounts of the prepared complexes (equivalent to 25 mg drug) (Table 1) and the results were compared with the low-loaded complexes (Table 6). The low- and high-loaded DEX HBr-EXPLOTAB® CLV complexes showed almost similar binding efficiency in all the extracting solvents examined Same pattern was achieved with DEX HBr-AC-DI-SOL® complexes except in normal saline and acetic acid solutions where the high-loaded drug-AC-DI-SOL® complex showed a decrease in the binding efficiency by more than 50% compared with the values obtained with the low-loaded complex. The contrast between the pattern of the results obtained with EXPLOTAB® and AC-DI-SOL® high-loaded complexes in normal saline and acetic acid solutions is likely due to ionic interference that had an impact on the drug-AC-DI-SOL® complex which is characterized by its very low polymer content (41.5 mg AC-DI-SOL® versus 214.3 mg EXPLOTAB®), rendering it more sensitive to ionic effect.

TABLE 6
Binding efficiency of EXPLOTAB ® CLV and AC-DI-SOL ® DEX HBr complexes in
different extracting solvents.

| | Low-loaded | High-loaded | Low-loaded | High-loaded |
|---|---|---|---|---|
| | EXPLOTAB ® CLV- | EXPLOTAB ® CLV | AC-DI-SOL ® | AC-DI-SOL ® |
| | Binding ± SD | | | |
| Water | 97 ± 0.2 | 93 ± 0.2 | 96 ± 0.2 | 94 ± 0.3 |
| pH 3 solution | 93 ± 0.3 | 90 ± 0.5 | 93 ± 0.2 | 88 ± 0.5 |
| Normal saline | 33 ± 0.7 | 27 ± 1.3 | 18 ± 1.1 | 6 ± 0.3 |
| 40% v/v ethanol | 94 ± 0.2 | 94 ± 0.0 | 93 ± 0.1 | 94 ± 0.4 |
| Acetic acid, 0.83M | 36 ± 0.9 | 37 ± 0.4 | 26 ± 0.5 | 5 ± 0.7 |
| Acetic acid, 0.5M | 44 ± 0.8 | 43 ± 0.4 | 33 ± 0.2 | 9 ± 0.5 |
| Acetic acid, 0.1M | 65 ± 0.1 | 61 ± 0.4 | 61 ± 0.2 | 26 ± 0.5 |

Drug release in 0.1 N HCl under therapeutic use: drug release under legitimate use was examined in vitro over one hour in 900 mL of 0.1 N HCl. The two complexes of each deterring agent (1:8 and 1:1.3 drug to polymer) were examined. The results (Table 7) indicate immediate and complete drug release from all complexes. The anionic EXPLOTAB® and AC-DI-SOL® were protonated upon contact with HCl dissolution medium and therefore DEX HBr was liberated from the formulation without any interference with the normal therapeutic use of the drug.

TABLE 7
Drug release from EXPLOTAB ® CLV- and AC-DI-SOL ®-drug complex tablets in 0.1N
HCl medium.

| | Low loaded | High loaded | Low loaded | High loaded |
|---|---|---|---|---|
| | DEX HBr- | DEX HBr- | DEX HBr- | DEX HBr- |
| | EXPLOTAB ® CLV | EXPLOTAB ® CLV | AC-Dl-SOL ® | AC-DI-SOL ® |
| Time | % Release ± SD | | | |
| 15 min. | 99.0 ± 0.68 | 101.8 ± 2.29 | 106.4 ± 1.38 | 101.7 ± 1.16 |
| 30 min. | 99.5 ± 0.35 | 106.0 ± 5.66 | 106.1 ± 1.84 | 101.4 ± 2.36 |
| 1 h. | 97.6 ± 0.99 | 104.3 ± 4.51 | 108.0 ± 4.33 | 100.4 ± 1.20 |

Drug re-binding in water and pH 7.5 phosphate buffer under therapeutic use: under regular conditions of drug use, the formulation will transit from the stomach to the intestine, exposing the drug to a higher pH value in the intestinal area. This might affect the protonation status occurring in the gastric medium (stage I dissolution) and thus re-binding between the polymer and drug may take place. If any re-binding occurs at this stage, it would adversely affect the therapeutic effectiveness of the drug, in particular, if the drug is formulated into an extended release dosage form. Stage II studies of dissolution over 24 hours, one in water and another in phosphate buffer of pH 7.5 were conducted on the complexes of EXPLOTAB® and AC-DI-SOL®. The results of % drug release in water dissolution medium (Table 8) show dissolution values within the USP acceptance criteria as per different monographs of opioid drugs. The results indicate that both EXPLOTAB® and AC-DI-SOL® are irreversibly protonated in simulated gastric fluid, therefore drug would be available for absorption in small and large intestine areas.

TABLE 8
% Drug release in EXPLOTAB ® CLV- and AC-DI-SOL ®-drug complexes in water as
dissolution medium.

| | Low loaded | High loaded | Low loaded | High loaded |
|---|---|---|---|---|
| | DEX HBr- | DEX HBr- | DEX HBr- | DEX HBr- |
| | EXPLOTAB ® CLV | EXPLOTAB ® CLV | AC-DI-SOL ® | AC-DI-SOL ® |
| Time | % Release ± SD | | | |
| 15 min. | 95.3 ± 1.43 | 98.3 ± 2.68 | 95.9 ± 0.82 | 106.7 ± 7.44 |
| 30 min. | 93.5 ± 1.58 | 98.5 ± 3.00 | 98.8 ± 0.96 | 102.9 ± 3.22 |
| 1 h. | 93.1 ± 0.32 | 98.5 ± 3.28 | 98.5 ± 0.99 | 101.1 ± 4.14 |
| 2 h. | 93.7 ± 1.16 | 94.3 ± 3.67 | 96.9 ± 1.17 | 100.3 ± 1.52 |
| 6 h. | 94.6 ± 1.54 | 93.9 ± 1.19 | 98.4 ± 1.39 | 96.8 ± 0.34 |
| 8 h. | 92.1 ± 1.24 | 92.9 ± 0.90 | 95.9 ± 0.50 | 98.9 ± 0.82 |
| 12 h. | 91.7 ± 0.70 | 92.7 ± 0.68 | 95.4 ± 1.32 | 96.9 ± 0.79 |
| 24 h. | 91.8 ± 0.74 | 92.7 ± 0.39 | 95.1 ± 1.08 | 98.2 ± 1.11 |

The results of drug concentration in pH 7.5 dissolution medium (Table 9) are also within the USP acceptance criteria for the four complexes. The irreversible protonation on one hand and the ionic effect derived from the ions of the phosphate buffer on the other hand plays major roles in maintaining the complete drug release initially attained in the acidic environment.

TABLE 9
% Drug release in EXPLOTAB ® CLV- and AC-DI-SOL ®-drug complexes in pH 7.5
phosphate buffer medium.

| | Low loaded | High loaded | Low loaded | High loaded |
|---|---|---|---|---|
| | DEX HBr- | DEX HBr- | DEX HBr- | DEX HBr- |
| | EXPLOTAB ® CLV | EXPLOTAB ® CLV | AC-DI-SOL ® | AC-DI-SOL ® |
| Time | Release ± SD | | | |
| 15 min. | 99.4 ± 2.49 | 94.8 ± 0.72 | 99.4 ± 0.81 | 98.7 ± 2.15 |
| 30 min. | 99.5 ± 2.22 | 95.3 ± 1.35 | 100.3 ± 0.59 | 98.7 ± 0.96 |
| 1 h. | 100.1 ± 2.47 | 96.1 ± 1.38 | 100.5 ± 2.08 | 97.9 ± 1.06 |
| 2 h. | 100.8 ± 2.89 | 93.9 ± 0.59 | 98.3 ± 0.39 | 97.7 ± 1.68 |
| 6 h. | 97.3 ± 0.84 | 97.2 ± 1.15 | 97.2 ± 1.04 | 97.2 ± 1.28 |
| 8 h. | 97.5 ± 4.02 | 96.9 ± 0.09 | 97.8 ± 0.36 | 96.9 ± 1.80 |
| 12 h. | 96.2 ± 2.83 | 93.5 ± 0.86 | 97.7 ± 0.22 | 96.8 ± 1.67 |
| 24 h. | 95.5 ± 2.39 | 93.9 ± 1.58 | 96.8 ± 1.46 | 95.5 ± 1.37 |

### Example 2: Minimize Drug Extraction Over a Wide Range of Extraction Temperatures

Although, most of the abuse-deterrent formulations available today more or less respond to abuse at room temperature, their deterrence mechanism can be significantly vulnerable at higher temperatures. Heat-activated drug abuse can be attempted either by heating the drug formulation in its solid or solution states, using either an open flame, electricity, or radiation. Our extensive studies on poly(ethylene oxide) (PEO), the widely-used deterrent agent in majority of ADFs, shows significant loss in polymer properties both in solution and solid states, which dramatically affects the deterrence capability of the dosage forms containing such polymer as major deterrent agent (Table 10).

TABLE 10
Summary of DSC study of heat-treated solid PEOs.

| | Weight, | Delta H, | Delta H/ | Peak, | X Onset, | Peak − Onset, | Area, |
|---|---|---|---|---|---|---|---|
| | mg | J/g | Wt. | ° C. | ° C. | ° C. | mJ |
| RT | 10.70 | 160.89 | 15.3 | 75.30 | 63.89 | 11.41 | 1721.56 |
| 80° C. | 15 | 102.27 | 6.81 | 72.48 | 63.05 | 9.43 | 1288.66 |
| 110° C. | 17 | 110.32 | 6.48 | 71.07 | 54.52 | 16.55 | 1268.70 |
| 150° C. | 23.5 | 122.61 | 5.21 | 65.20 | 43.45 | 21.75 | 1594.00 |
| 150° C.-3 hrs | 12.8 | 93.03 | 7.26 | 54.89 | 47.57 | 7.32 | 93.03 |
| 180° C. | 15.3 | 91.42 | 5.97 | 54.65 | 45.85 | 8.8 | 1737.11 |

A 5% w/v PEO (WSR coagulant) solution in water at 90° C. was 80% less viscous (1400 cP to 300 cP) as measured at 100 sec⁻¹ for 40 sec. using Brookfield cone and plate rheometer DV-III, Ultra) than the same solution at room temperature. Solid PEO (WSR coagulant) turns off-white and suffers from oxidative degradation at higher temperatures. Reduced aqueous solution viscosity was observed for the pre-heated PEO (WSR coagulant) in powder form that was heated at 80, 110, 150 and 180° C. for 1 hour in a hot air oven. After cooling to room temperature, heat-treated PEOs were used to prepare 2% w/v solutions. The same solution, prepared using non-heated sample, was used as control. The viscosity of all solutions was measured using a cone and plate rheometer (Brookfield DV-III Ultra) at a shear rate of 300 sec⁻¹ for 40 sec. Results are shown in Table 11 where there was a drop in aqueous viscosity of solid PEO (WSR coagulant) with increasing temperatures (dramatically between 110° C. and 150° C.).

TABLE 11
Aqueous viscosities of PEO pre-heated at different temperatures.

| | Viscosity | Viscosity | Viscosity | |
|---|---|---|---|---|
| Sample | Run1 | Run2 | Run3 | Mean ± SE |
| Room | 383.9 | 373.96 | 377.23 | 378.36 ± 2.388 |
| Temp | | | | |
| @ 80° C. | 294.3 | 285.8 | 296.92 | 292.34 ± 2.74 |
| @ 110° C. | 221.71 | 224.98 | 225.63 | 224.10 ± 0.99 |
| @ 150° C. | 3.92 | 5.89 | 4.15 | 4.65 ± 0.50 |
| @ 180° C. | 3.27 | 4.58 | 3.27 | 3.70 ± 0.35 |

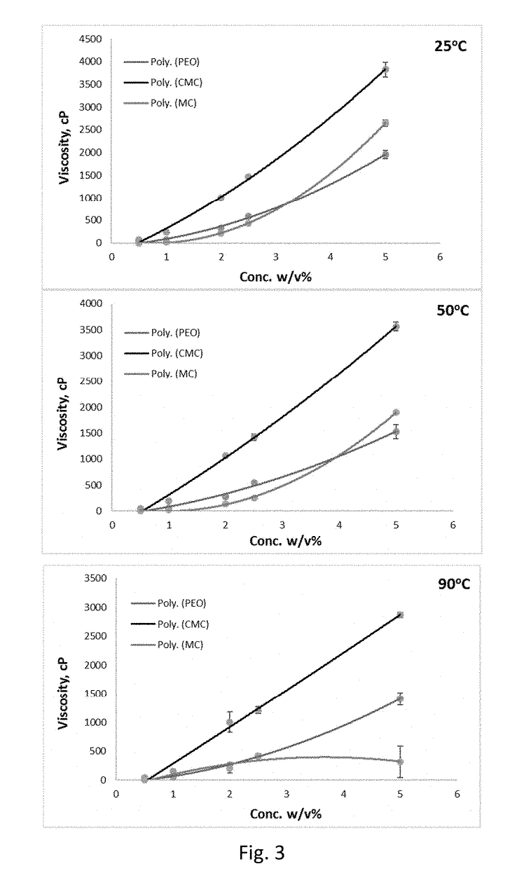

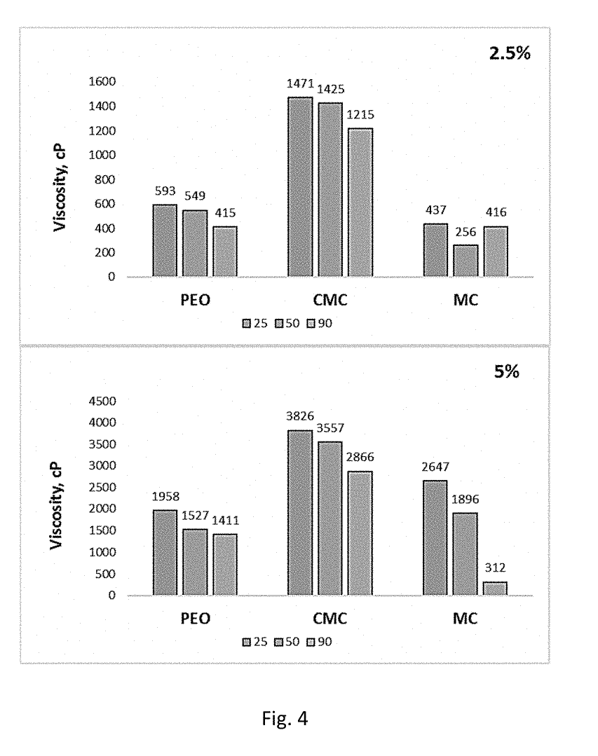

The solution properties of two cellulosic polymers [methylcellulose (MC) and carboxymethylcellulose (CMC)] are either independent of the solution temperature or are activated or enhanced at higher temperatures. Using the two polymers in a formulation can minimize drug extraction if attempted over a wide temperature range. To study the effect of solid concentration and temperature on viscosity, different concentrations (0.5, 1, 2, 2.5, and 5 w/v %) of PEO, CMC (TICALOSE® CMC 15), and MC (METHOCEL™ A4C) were prepared, and their viscosities were measured using a Brookfield Cone & Plate rheometer (DV Ultra III) at room temperature, 50° C. and 90° C. At all concentrations (0.5-5 w/v %), PEO and CMC solutions display lower viscosities at higher solution temperatures, however, the MC solution displayed an anomaly at 90° C. As shown in FIG. 3, CMC solutions display significantly higher viscosity values than PEOs at all temperatures (25, 50, and 90° C.), however the same anomaly was observed for MC. As shown in FIG. 4, CMC solutions display significantly higher viscosity values than PEOs at low and high temperatures, however MC viscosity was lower due to the anomaly. An investigation into the MC anomaly revealed that the rheometer software was unable to read the viscosity values of the MC solutions, in particular at higher MC solutions and at higher temperatures, due to heat-activated gel formation, i.e., thermogelation.

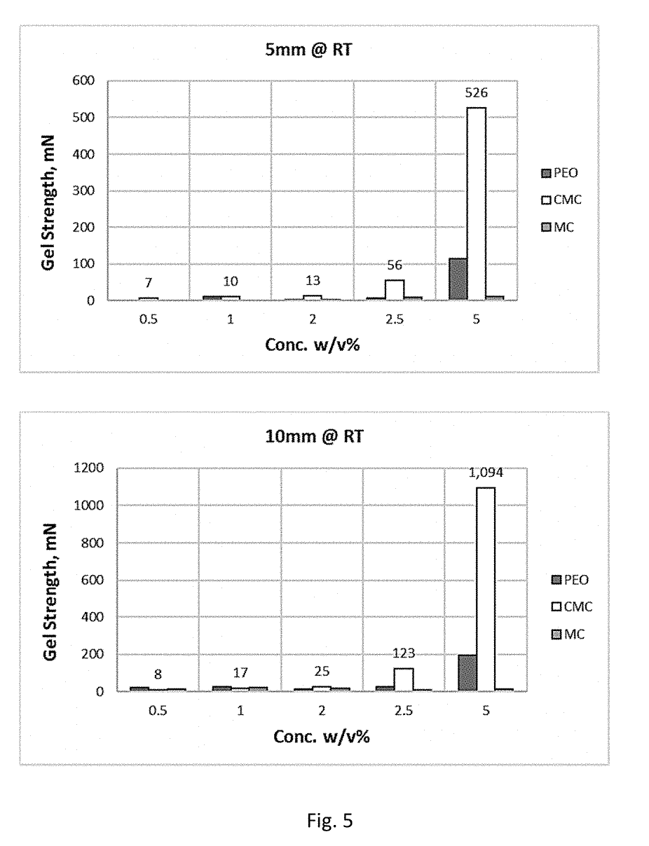

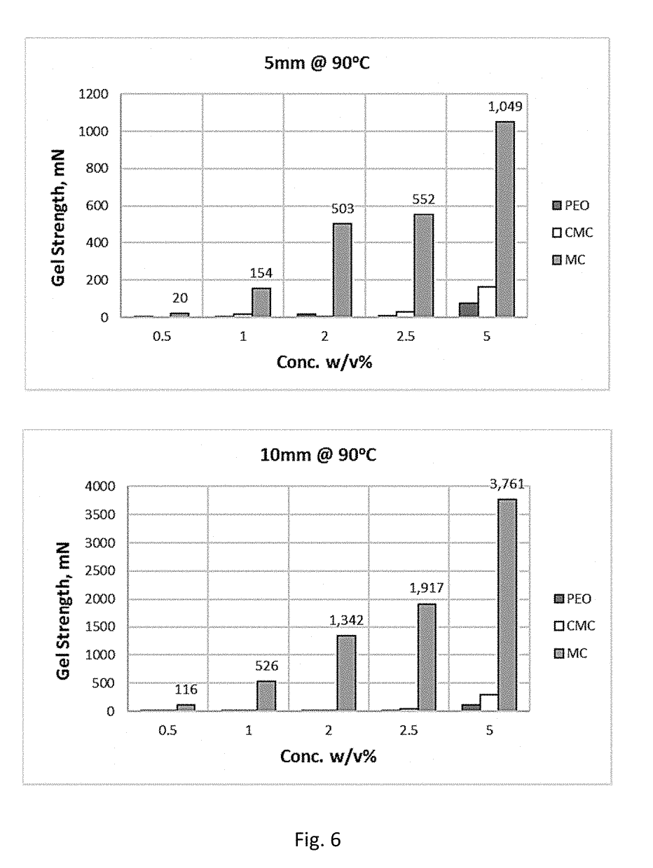

Gel strength—a CT3 Texture Analyzer was used to measure gel strength of the PEO, CMC, and MC solutions at room temperature and 90° C. A jacketed beaker was attached to a circulating water bath maintained at 90° C. Each solution was poured and left in the beaker for 5 min. Gel strength was then measured by allowing the texture analyzer probe to travel into the solution up to 10 mm distance. The resistance exercised by the solution, as the probe travels into the solution, indicates gel strength of the solution measured in mN. The speed of probe was set at 1 mm/sec. Results are shown in FIGS. 5 and 6.

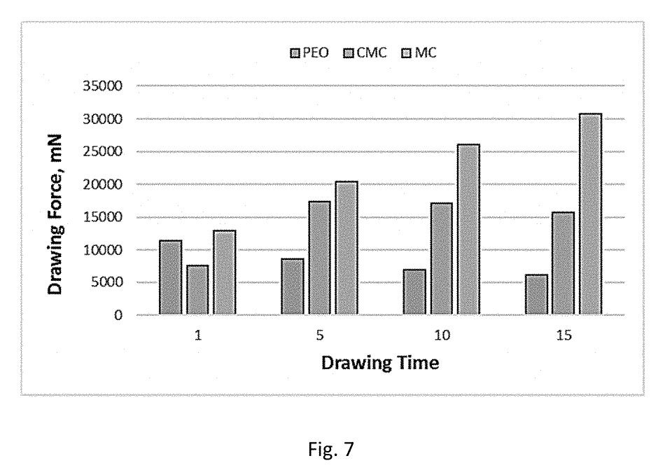

Stress History—this test determines the ease at which the solution can be drawn up into the syringe after it undergoes multiple aspirations, pulling in and out of syringe, or so-called stress history. About 3 mL of 1% w/v solutions of PEO, CMC, and MC were taken into glass vials. A Brookfield Conn.3 texture analyzer with syringe probe was used to measure the pulling (drawing) force. The syringe plunger was attached to the probe while syringe barrel with the needle was immersed into the solution. The solution was drawn up into the syringe up to 3 mL mark by pulling the probe up. The probe was then pushed down to release the solution into glass vial. The probe was pulled again and solution was drawn up to the 3 mL mark. This procedure was repeated 15 times and the force for pulling the probe was recorded each time. The drawing force for the solutions was compared at first, fifth, tenth, and fifteenth pulling in/out cycles. As shown in FIG. 7, after 1, 5, 10, and 15 cycles of pulling in/out, the MC solutions show dramatically better endurance to stress history. As the number of drawing increases, the drawing force increases for MC solution, decreases for PEO solution, and remains relatively unchanged with CMC solution.

Aspirated Volume—syringeability study measures the force required to aspirate the solution into syringe. The solutions of PEO, CMC and MC were prepared at 0.5, 1, 2, 2.5, and 5% w/v concentrations. A CT3 Texture Analyzer with a syringe probe was used to draw solution up into the syringe. The syringe plunger was attached to the probe and the needle was immersed into the solution. The probe was pulled up to 40 mm mark at the speed of 0.5 mm/sec. Although the force required to draw solution into the syringe was same for all samples, the aspirated volume after 1 min was different. As shown in Table 12, at 0.5-1 w/v % solution concentrations, all three polymers behaved similarly showing maximum aspirated volume of 2, same obtained for PEO at 2 w/v % concentration, however the aspirated volume decreased significantly with the MC solutions at higher concentrations, and no volume was aspirated from the CMC solutions at greater than 2 w/v % concentrations.

TABLE 12
Volume (mL) aspirated into the syringe at low solution temperature.

| Concentration, | | | |
|---|---|---|---|
| w/v % | PEO | CMC | MC |
| 0.5 | 2 | 2 | 2 |
| 1 | 2 | 2 | 2 |
| 2 | 2 | 0 | 0.5 |
| 2.5 | 1.5 | 0 | 0.3 |
| 5 | 0 | 0 | 0 |

To measure aspirated volume at higher temperature, a jacketed glass beaker was attached to a water bath maintained at 90° C. The sample solutions, prepared as mentioned above, were poured into the beaker. After 5 min, the temperature of the sample solution and the aspirated volume were measured. Results as shown in Table 13.

TABLE 13
Volume (mL) aspirated into the syringe
at high solution temperature.

| Concen- | PEO | | CMC | | MC | |
|---|---|---|---|---|---|---|
| tration, | | Volume, | | Volume, | | Volume, |
| w/v % | T, ° C. | mL | T, ° C. | mL | T, ° C. | mL |
| 0.5 | 65 | 3 | 62 | 3 | 66.5 | 0 |
| 1 | 73 | 3 | 60 | 3 | 65 | 0 |
| 2 | 74 | 2.5 | 61 | 0.2 | 73 | 0 |
| 2.5 | 70 | 1.5 | 63 | 0 | 72 | 0 |
| 5 | 72 | 0 | 62 | 0 | 71 | 0 |

At 0.5-1 w/v % solution concentrations, PEO and CMC behaved similar with maximum aspirated volume of 3 mL, aspirated volume steadily decreased for PEO at concentrations greater than 2 w/v %, aspirated volume for CMC decreased significantly at solution concentrations greater than 2 w/v %; and at all concentrations, no volume was aspirated from the MC solutions.

### Example 3: Minimizing Drug Extraction Using In-Situ Gelling Polymers

Crosslinked poly(acrylic acid) (CARBOPOL®) and its fine combination with sodium bicarbonate can be incorporated into an abusable dosage form, providing improved gelling properties over PEO-based formulations. These new formulations can deter drug abuse by effectively binding the drug in solution, a property which is not offered by PEO.

Dextromethorphan HBr USP was obtained from Letco medical LLC, AL, USA), CARBOPOL® (940) was obtained from Acros Organics, NJ, USA, PEO grades (Mw 100,000 and POLYOX™ WSR coagulant) were obtained from Sigma-Aldrich Co., MO, USA and Colorcon Ltd., PA, USA, respectively, and AVICEL® (PH 102) and sodium bicarbonate USP #2 were obtained from FMC corporation, PA, USA. Ethanol (Decon labs Inc., PA, USA) and hydrochloric acid (Merck, Germany) used were of analytical grade.

Tablets were prepared by direct compression. All ingredients shown in Table 14 were sifted through a mesh #60 sieve, and mixed thoroughly to ensure uniformity. The tablets were prepared using a single station compression press (Carver Inc., IN, USA) with ½ inch diameter standard concave tooling (EC #1 08-14) at the compression force of 2000 Lb.

TABLE 14
Tablet formulations used in preliminary studies.

| Ingredient | CTRL 1 | CTRL 2 | Sample |
|---|---|---|---|
| Dextromethorphan HBr | 25 | 25 | 25 |
| CARBOPOL ® | — | 250 | 250 |
| Sodium bicarbonate | — | — | 50 |
| AVICEL ® | 475 | 225 | 175 |
| Total | 500 | 500 | 500 |

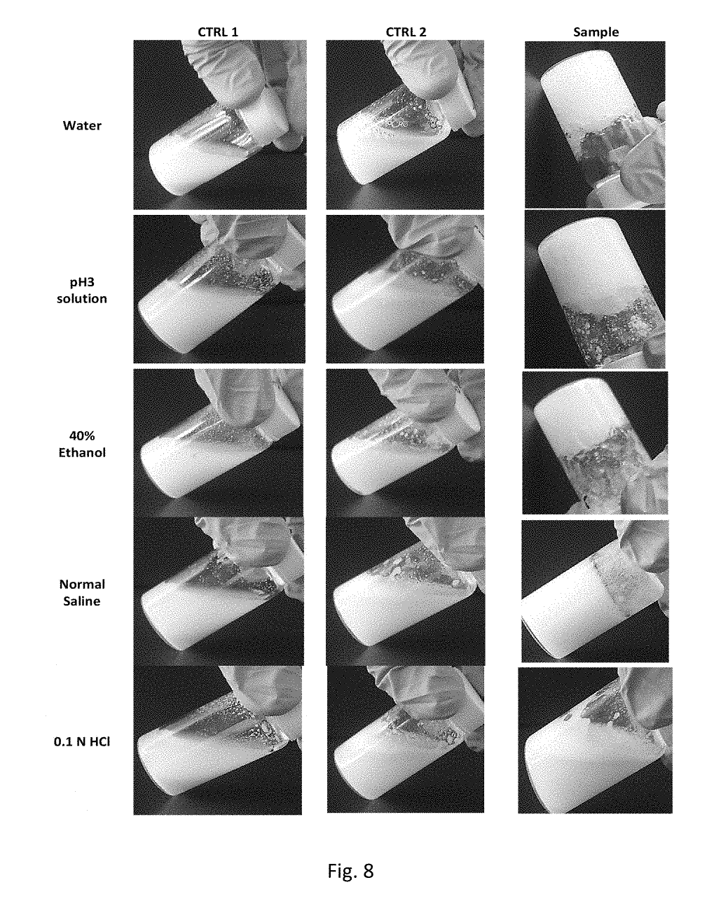

Extraction studies. Prepared tablets were crushed, and drug extraction was attempted under vortex-mixing in small volume (10 mL) of solvent (water, pH 3, 40% ethanol, normal saline, and 0.1N HCl) to simulate an IV abuse and to demonstrate the behavior of the formulation in simulated acidic environment of the stomach. The mixture formed gel in all solvents almost immediately (within 30 sec of stirring) except in 0.1 N hydrochloric acid. In saline, the gel was not formed instantly, but formed with some delay. As shown in FIG. 8, the tablet formulation containing 250 mg CARBOPOL® and 50 mg sodium bicarbonate turns the liquid extraction solution into gel in all extraction media studied. In 0.1N HCl, the sodium bicarbonate reaction with the acid leaves CARBOPOL® with no gelling agent and hence no gel formation, which is a desirable feature in abuse-deterrent formulations.

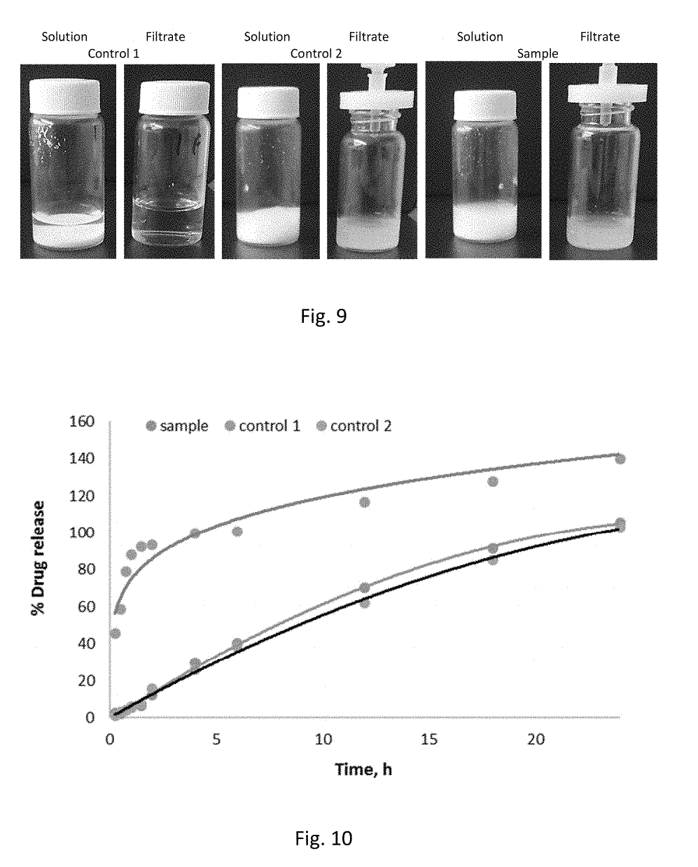

Filtering the extraction solution. Formulations dispersed in 10 mL of 0.1 N HCl were centrifuged at 1500 RPM for 5 min. The supernatant was collected and filtered through a 0.2 μm syringe filter. The filtrate was analyzed using UV-Vis spectroscopy to determine the amount of active drug in the solution. With formulations containing CARBOPOL® (control 2 and sample), the polymer passed through the filter, and therefore such formulations are not filterable (FIG. 9). The amount of drug in each filtrate was measured using UV-Vis spectrophotometer (Shimadzu, UV-1700) at 276 nm as shown in Table 15, however these results are not confirmatory as CARBOPOL® particles in the filtrate interfered with the spectrophotometer readings.

TABLE 15
% Drug in filtrates.

| | CTRL 1 | CTRL 2 | Sample |
|---|---|---|---|
| AVG (n 4) | 1.248 | 1.683 | 1.798 |
| Conc. | 240.077 | 323.567 | 345.755 |
| DF | 2400.77 | 3235.67 | 3457.55 |
| mg/mL | 2.401 | 3.2357 | 3.4576 |
| % | 96.03 | 129.43 | 138.30 |

Dissolution Studies. Dissolution study was carried out in USP type II (Paddle) dissolution apparatus at 50 RPM using 900 mL of 0.1 N hydrochloric acid at 37±0.5° C. The amount of drug released was determined by collecting the sample solution at specific time intervals and analyzing the dissolutions samples using a UV-Vis spectrophotometer (Shimadzu, UV-1700) at 276 nm. Drug release profile was obtained by plotting % drug released from the formulation against time as shown in FIG. 10. Formulations containing CARBOPOL® with and without sodium bicarbonate (SBC) behaved similar and released drug in a zero-order fashion over 24 hours dissolution period.

Formulation optimization: Formulation was optimized based on extraction and gel formation behavior in all solvents. With a same 5:1 weight ratio of CARBOPOL®/SBC, five formulations were prepared containing different amounts of CARBOPOL® ranging 10-40 wt % of the formulation. The amount of CARBOPOL® in preliminary formulation was 50 wt %. This study was aimed at reducing CARBOPOL® content of the formulation, while still providing effective abuse-deterrent properties. New formulations composed of and SBC were prepared at different CARBOPOL®/SBC concentrations as shown in Table 16.

TABLE 16
New formulations.

| Ingredient | Batch 1 | Batch 2 | Batch 3 | Batch 4 | Batch 5 |
|---|---|---|---|---|---|
| CARBOPOL ® | 50 | 100 | 125 | 150 | 200 |
| Sodium bicarbonate | 10 | 20 | 25 | 30 | 40 |
| AVICEL ® | 440 | 380 | 350 | 320 | 260 |
| Total | 500 | 500 | 500 | 500 | 500 |

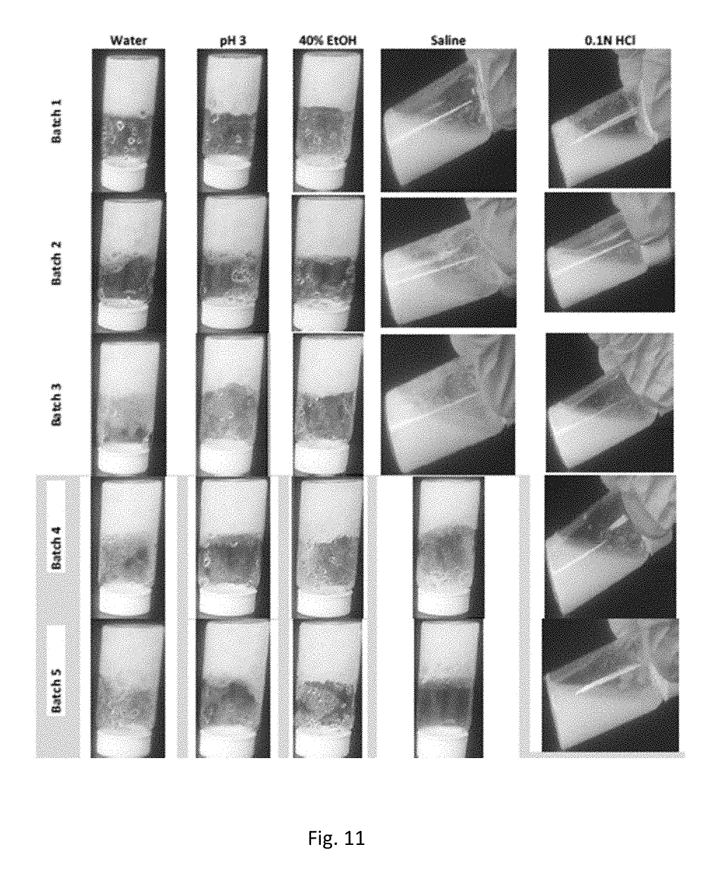

Extraction Studies: New formulations were subjected to 10 mL extraction in different solvents including water, pH3 solution, 40% ethanol, normal saline, and 0.1N hydrochloric acid. As shown in FIG. 11, a tablet formulation (500 mg) containing at least 150 mg of CARBOPOL® forms an effective gel in all extraction media, while maintaining its liquid state in 0.1N HCl medium.

Gel strength: A quantitative method was used to characterize gel formation in all extraction solvents. 10 mL of extraction solvent was added to the formulations in a glass vial. The contents were vortex mixed and subjected to gel strength measurement using a texture analyzer (Brookfield, Conn.3-4500). A resistance sensitive probe, attached to texture analyzer, was allowed to travel into gel up to 5 mm and 10 mm distances at the rate of 1 mm/sec. The resistance exerted by the gel (in mN) was then measured by the software as shown in Tables 17 and 18 for the target distance of 5 and 10 mm, respectively. This study confirms that an effective gel mass is formed in all extraction solutions including normal saline when the formulation contains at least 150 mg of CARBOPOL® (Formulations B4 and B5). Based on extraction and gel strength measurement studies, Formulation B4 (150 mg CARBOPOL®+30 mg SBC) was selected for further evaluations.

TABLE 17
Gel strength of the new formulations at target distance of 5 mm.

| Batch | Water | | pH 3 | | ethanol | | Normal saline | |
|---|---|---|---|---|---|---|---|---|
| B1 | 165.3 | (±7.6) | 108 | (±5) | 73.3 | (±9) | — | |
| B2 | 202.3 | (±8.1) | 176.6 | (±3.6) | 111.3 | (±6.2) | — | |
| B3 | 220.6 | (±7.7) | 194.3 | (±6.2) | 118 | (±4) | — | |
| B4 | 283 | (±5.6) | 214.3 | (±10.2) | 130.6 | (±5.7) | 209.3 | (±2.3) |
| B5 | 297.3 | (±15.4) | 246.6 | (±4.7) | 143.6 | (±9.4) | 214 | (±4.9) |

TABLE 18
Gel strength of the new formulations at target distance of 10 mm.

| Batch | Water | | pH 3 | | ethanol | | Normal saline |
|---|---|---|---|---|---|---|---|
| B1 | 186.3 | (±5) | 145.6 | (±7.7) | 91.3 | (±10.4) | — |
| B2 | 265 | (±10) | 213 | (±4.9) | 145 | (±5.8) | — |
| B3 | 294.3 | (±4.1) | 252 | (±2.1) | 173.3 | (±8.1) | — |
| B4 | 325.6 | (±8.3) | 264.6 | (±11.8) | 178 | (±4.2) | 216.7 (±3.2) |
| B5 | 351.6 | (±7.8) | 321 | (±5.8) | 197.6 | (±4.7) | 287.3 (±4.7) |

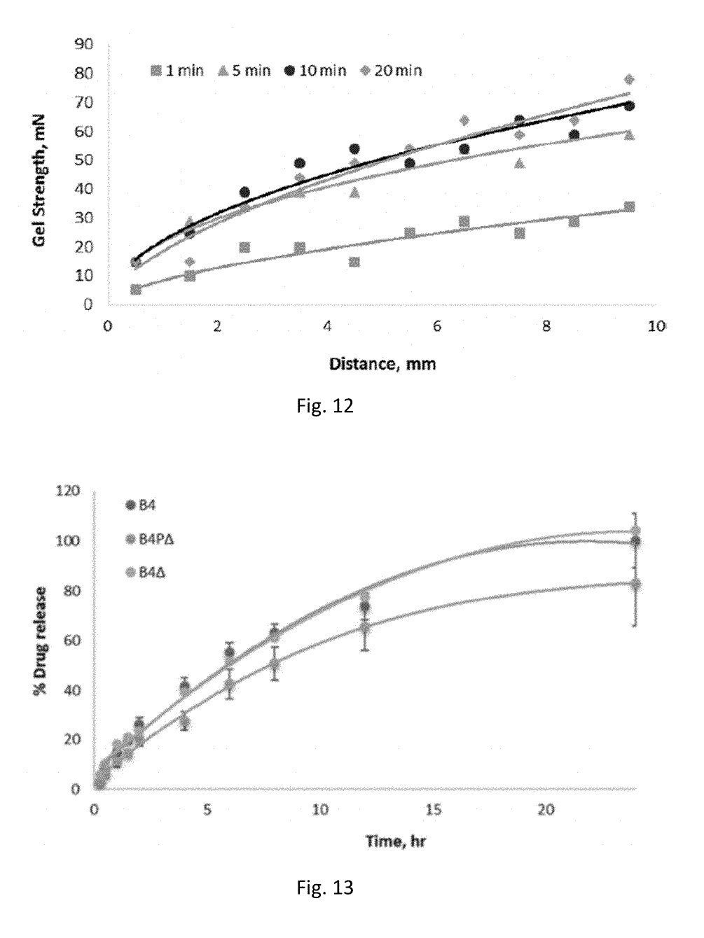

Gelation time in normal saline solution: To assess the capability of the formulation to deter abuse in a reasonable time, the lag period was determined by measuring the gel strength at certain time intervals using a texture analyzer (Brookfield, Conn.3-4500). A normal saline solution (10 mL) was added to formulation B4 and gel strength of the solution was measured after 1, 5, 10, and 20 min time interval. The probe was allowed to travel into gel up to 10 mm distance at the rate of 0.5 mm/sec. The resistance exerted by the gel (in mN) was plotted against the distance travelled by the probe (FIG. 12). This study shows that formulation B4 containing 150 mg CARBOPOL® and 30 mg sodium bicarbonate starts forming a reasonable gel in at least 5 minute and preferably 10 minutes after the mixture is placed in saline. Gel strength data after 10 and 20 minutes are statistically similar.

Crush resistance: Formulation B4 containing 150 mg CARBOPOL® and 30 mg sodium bicarbonate offered very effective extraction resistance properties in water, pH 3, 40% ethanol, and saline. Such formulation has a strong potential to effectively deter abuse by injection. In order to enhance crush resistance properties of this formulation, 150 mg of poly(ethylene oxide) (molecular weight 100,000 Da) was added to the B4 formulation. The tablet was subjected to heat treatment in a hot air oven at 80° C. for 30 min. Tablets without PEO and tablets with no heat treatment were used as control as shown in Table 19.

TABLE 19
Formulations for crush resistance studies.

| Ingredient | B4 | B4Δ | B4PΔ |
|---|---|---|---|
| Dextromethorphan HBr | 25 | 25 | 25 |
| CARBOPOL ® | 150 | 150 | 150 |
| Sodium bicarbonate | 30 | 30 | 30 |
| PEO | — | — | 150 |
| AVICEL ® | 295 | 295 | 145 |
| TOTAL | 500 | 500 | 500 |
| Heat treatment, 80° C. for | | ✓ | ✓ |
| 30 min | | | |

Dissolution study: The tablets listed in Table 19 were subjected to dissolution testing in 0.1 N HCl using a USP type II (Paddle) dissolution apparatus at 50 RPM. The dissolution analysis was carried out using a UV-Vis spectrophotometer (Shimadzu, UV-1700) at 276 nm. The dissolution profile was obtained by plotting % drug release against time for B4 formulation at room temperature (B4), B4 heat-treated at 80° C. (B44), and heat-treated B4 containing PEO (B4P4) as shown in FIG. 13. This study shows that heat treatment on formulation B4 has no effect on its dissolution profile. However, the same heat-treated formulation containing PEO displayed incomplete release.

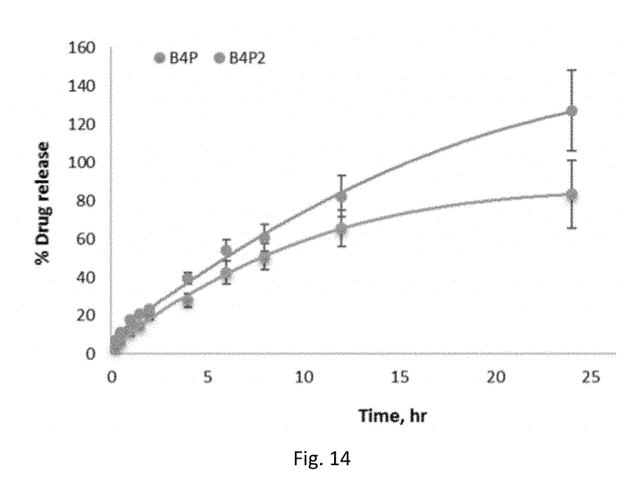

Another formulation containing lower amounts of PEO (CARBOPOL®/PEO ratio of 1.5) was evaluated (Table 20), and its dissolution profile is as shown in FIG. 14. Formulation B4P2 (containing 100 mg PEO) offers more drug release than Formulation B4P (containing 150 mg PEO).

TABLE 20
Formulation containing lower amount of PEO.

| Ingredient | B4P2 |
|---|---|
| Dextromethorphan HBr | 25 |
| CARBOPOL ® | 150 |
| PEO | 100 |
| Sodium bicarbonate | 30 |
| AVICEL ® | 195 |
| TOTAL | 500 |

Heat treatment, 80° C. for 30 min

Crush resistance: Both B4 and B4P formulations were subjected to crush resistance studies. Additionally, B4 tablets were heat-treated and similarly subjected to crush resistance tests. B4P tablets with no heat treatment were also tested for crush resistance. One tablet from each formulation set was subjected to crushing using an industry grade high shear grinder (MicroMill® II, Scienceware Inc.) with ½ hp, 150 watts (Table 25), and a domestic blender with sharp stainless-steel cross blades attached to a high torque power base (250 W, 60 Hz, 120 V) (Table 20) for 1 min. The crushed tablets were studied for particle size distribution.

Particle size distribution: The particle size distribution was determined by the weight retained on sieves stacked in a column by sieve number (20, 35, 60, 120, and 325 mesh equivalent to particle sizes of 850, 500, 250, 125 and 45 μm, respectively). Samples were placed into the top sieve, and the loaded sieve shaker (Cole Parmer SS-3CP) was set to tap for 1 min at 60 taps/min prior to weighing. The particle size distribution of tablet crushed by high shear grinder and domestic blender indicates higher percentage of coarser particles for heat-treated formulations containing PEO. Particle size distribution data also suggests that PEO addition and heat treatment generates very low percentage of desirable particle size (typically 150 μm and lower) for snorting or insufflation.

TABLE 21
High shear grinder study on B4 and B4P tablets (regular versus heat treated).

| | Particle | % retained | | | | | | | |
|---|---|---|---|---|---|---|---|---|---|
| Sieve | size | B4 | | | | B4P | | | |
| number | (μm) | Room temp | | Heated @ 80° C. | | Room temp | | Heated @ 80° C. | |
| 20 | >850 | 11.72 | (±0.4) | 29.43 | (±2.4) | 34.18 | (±1.0) | 79.19 | (±1.5) |
| 35 | 850-500 | 26.09 | (±0.3) | 26.96 | (±1.9) | 26.94 | (±0.9) | 10.71 | (±0.9) |
| 60 | 500-250 | 25.46 | (±0.7) | 22.15 | (±0.5) | 21.80 | (±0.9) | 5.25 | (±0.3) |
| 120 | 250-125 | 22.27 | (±0.3) | 11.96 | (±1.2) | 9.33 | (±0.5) | 2.55 | (±0.1) |
| 325 | 125-45 | 10.73 | (±0.6) | 8.53 | (±2.0) | 5.78 | (±0.2) | 1.93 | (±0.5) |
| >325 | <45 | 3.70 | (±0.9) | 0.94 | (±0.6) | 1.94 | (±0.2) | 0.34 | (±0.2) |

Particle size distribution data for the formulation containing PEO (B4P) showed higher percentage of larger particles compared to other formulations. With heat treated B4P formulation, approximately 80% of the particles were larger than 850 μm whereas for formulation without PEO, particles were more uniformly distributed across all size ranges. This shows that B4P formulation with heat treatment has higher resistance to breakdown into smaller fragments. This property in particular is useful to deter abuse via nasal route, where desired particle size is smaller than or equal to 150 μm.

TABLE 22
Domestic blender study on B4 and B4P tablets (regular versus heat treated).

| | Particle | % retained | | | | | | | |
|---|---|---|---|---|---|---|---|---|---|
| Sieve | size | B4 | | | | B4P | | | |
| number | (μm) | Room temp | | Heated @ 80° C. | | Room temp | | Heated @ 80° C. | |
| 20 | >850 | 13.49 | (±0.4) | 3.71 | (±1.4) | 3.83 | (±1.0) | 56.09 | (±0.6) |
| 35 | 850-500 | 28.57 | (±0.3) | 25.98 | (±2.0) | 22.17 | (±1.9) | 19.30 | (±1.4) |
| 60 | 500-250 | 27.38 | (±0.6) | 33.18 | (±0.9) | 42.64 | (±1.2) | 14.83 | (±1.3) |
| 120 | 250-125 | 13.49 | (±0.2) | 17.24 | (±0.2) | 17.27 | (±0.9) | 7.31 | (±1.7) |
| 325 | 125-45 | 11.70 | (±0.5) | 16.37 | (±1.2) | 11.30 | (±1.0) | 3.86 | (±0.9) |
| >325 | <45 | 5.35 | (±0.9) | 3.49 | (±0.2) | 2.77 | (±0.7) | 0.60 | (±0.1) |

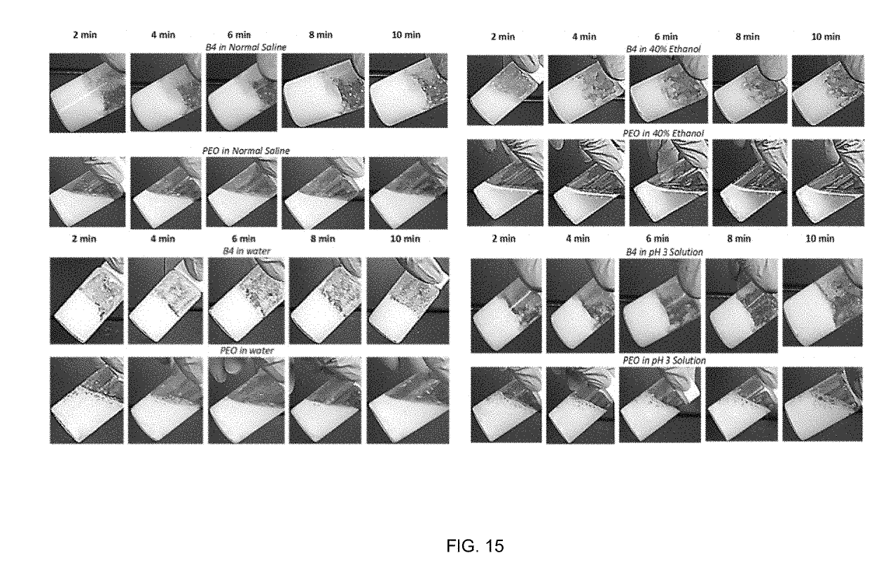

Advantage of new formulation over PEO-based formulations—instant gelation. Into separate 20 mL glass vials containing B4 and PEO formulations (Table 23), 10 mL of solvents were added and the contents were vortex mixed for 30 sec. The tilted vials were photographed every 2 minutes for 10 minutes. In all solutions, the B4 formulation formed an instant solid gel while the PEO formulations stayed as liquid even after 10 minutes (FIG. 15).

TABLE 23
B4 and PEO Formulations.

| B4 Formulation | Amount, mg | PEO Formulation | Amount, mg |
|---|---|---|---|
| Dextromethorphan | 25 | Dextromethorphan | 25 |
| HBr | | HBr | |
| CARBOPOL ® | 150 | PEO (WSR, | 150 |
| | | coagulant) | |
| Sodium bicarbonate | 30 | AVICEL ® | 325 |
| AVICEL ® | 295 | TOTAL | 500 |
| TOTAL | 500 | | |

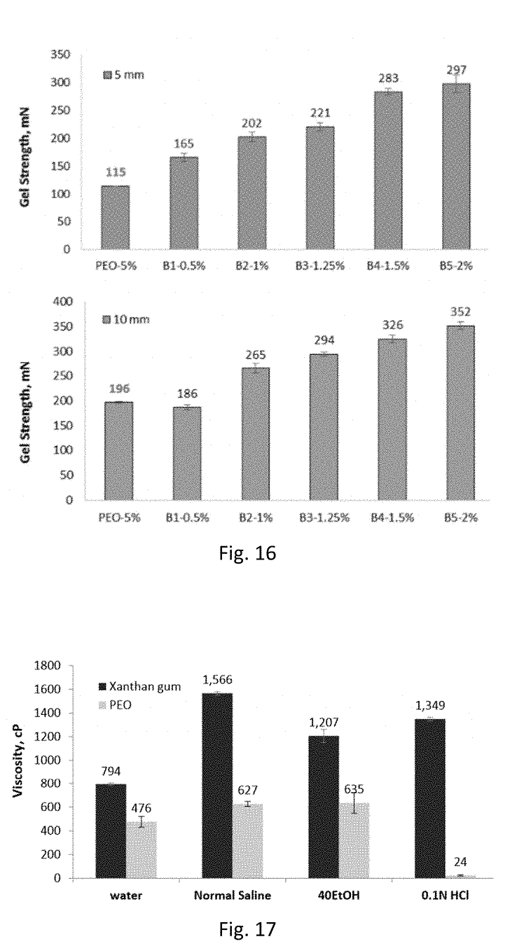

High equilibrium gel strength: Gel strength of PEO solution (5 wt %) and all new formulations containing different amounts of CARBOPOL® (0.1-2%) were measured after 24 hrs. FIG. 16 shows that the gel formed in 10 mL of water by formulation B4 at 1.5 wt % CARBOPOL® concentration is 1.5-2.5 times stronger than the gel formed by high molecular weight PEO at 5 wt % concentration.

### Example 4: Minimizing Drug Extraction Utilizing Rigid Stable Polymers

The use of bacterial gums such as xanthan gum that can provide more effective abuse deterrence properties compared to poly(ethylene oxide) compositions due to swelling, gel forming and binding. Majority of poly(ethylene oxide) based formulations suffer from lack of performance under severe extraction conditions especially in the presence of salt and alcohol. In this study, we show that using xanthan gum in abuse deterrent formulations provides enhanced deterrence capacity under variety of abuse conditions.

Viscosity comparison of xanthan gum and PEO. Dextromethorphan HBr USP (Letco medical LLC, AL, USA) was used as a model drug. Xanthan gum and poly(ethylene oxide) were obtained from TIC gums Inc., MD, USA and Colorcon Inc., PA, USA, respectively. AVICEL® PH 102 was obtained from FMC corporation, PA, USA. Ethanol (Decon labs Inc., PA, USA) and hydrochloric acid (Merck, Germany) were of analytical grade. Solutions of xanthan gum (1 wt %) and PEO (1 wt %) were prepared in different solvents including water, 0.1N hydrochloric acid, 40% v/v ethanol (aq), and normal saline. Xanthan gum and PEO (each 100 mg) were added into 10 mL of solvents and allowed to hydrate completely. Using a cone and plate rheometer (Brookfield, DV-III, Ultra), the viscosities of hydrated solutions were measured at a shear rate of 100 sec-1 for 40 sec as shown in FIG. 17.

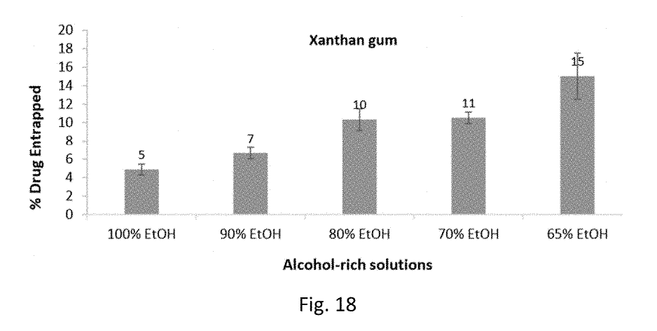

Drug entrapment via binding (complexation): Because of very high viscosity and lack of filterability of aqueous solutions of xanthan gum, its extraction and drug entrapment was measured in alcohol rich solutions by gradually increasing the amount of water. For this purpose, drug was dissolved in 10 mL solutions at different ethanol concentrations including 100%, 90%, 80%, 70% and 65% v/v. 250 mg of xanthan gum was then added to each solution. Similarly, xanthan gum solutions without addition of drug were prepared and used as control. After centrifugation for 5 min at 1500 rpm and filtration using 0.2 μm syringe filter, the amount of drug entrapped was indirectly calculated by measuring the amounts of drug remaining in the filtrate using a UV-Vis spectrophotometer (Shimadzu, UV-1700) at a wavelength of 276 nm, data shown in FIG. 18. The resulting data showed that xanthan gum is capable of forming a complex with weak bases.

Tablet formulations: A tablet formulation containing xanthan gum was prepared. Another formulation with additional poly(ethylene oxide) (PEO, MW 100,000 Da) was also prepared and heat-treated for 30 min at 80° C. to incorporate crush resistance properties to the tablet. Tablets were prepared by direct compression. All ingredients (shown in Table 24) were sifted through a mesh #60 sieve and mixed thoroughly to ensure uniformity. The tablets were prepared using a single station compression press (Carver Inc., IN USA) with ½ inch diameter standard concave tooling (EC #1 08-14) at the compression force of 2000 Lb.

TABLE 24
Tablet Formulations.

| Ingredient | Formulation 1, mg | Formulation 2, mg |
|---|---|---|
| Dextromethorphan HBr | 25 | 25 |
| Xanthan gum | 250 | 250 |
| PEO | — | 100 |
| AVICEL ® (PH 102) | 125 | 25 |
| Heat treatment @ 80° C. | — | ✓ |
| for 30 min | | |
| TOTAL | 400 | 400 |

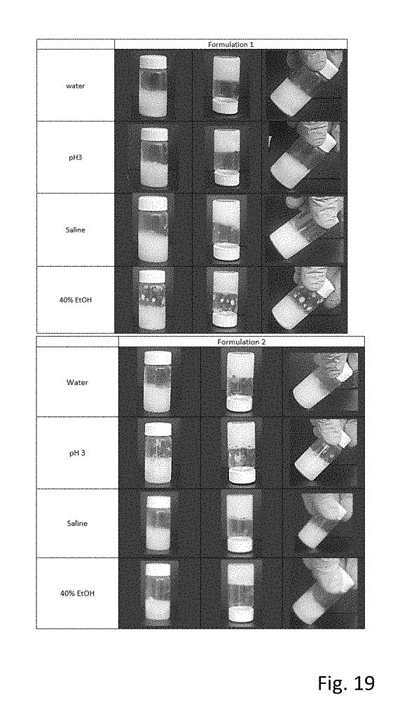

Extractability and gel forming behavior: To study extractability and gel forming behavior under simulated ‘worst-case’ extraction conditions, the formulation was exposed to extraction in 10 mL of different solvents. The solvents included water, 40% v/v ethanol, pH3 solution, normal saline, and 0.1N hydrochloric acid. The formulation formed very strong gels in all solvents except in 0.1N hydrochloric acid (FIG. 19).

Gelation time in normal saline: The gelation in normal saline was observed with a time-lag. To study time-lag gelation behavior, the gel strength was measured after specified time intervals as shown in Table 25.

TABLE 25
Gelation time in normal saline.

| Time, h | Gel strength, mN |
|---|---|
| 0 | 0 |
| 0.08 | 15 |
| 0.25 | 25 |
| 0.5 | 25 |
| 1 | 34 |
| 2 | 34 |
| 3 | 39 |
| 18 | 39 |
| 24 | 44 |

Gel strength measurement: A quantitative method was used to characterize gel formation in all extracting solvents. 10 mL of solvent was added to the formulations in a glass vial. The contents were vortex mixed and allowed to stand for 10 min. Gel strength measured using a texture analyzer (Brookfield, Conn.3-4500). A resistance sensitive probe, attached to texture analyzer, was allowed to travel into gel up to 5 and 10 mm distances at the rate of 0.5 mm/sec. The resistance exerted by the gel was measured in mN using the software as shown in Tables 26 and 27.

TABLE 26
Gel strength (mN) of formulation 1.

Target distance

| Solvent | 5 mm | 10 mm |
|---|---|---|
| Water | 45.6 ± 2.3 | 54.0 ± 4 |
| pH 3 | 47.3 ± 2.3 | 57.3 ± 9.4 |
| 40% v/v hydroalcoholic | 45.6 ± 2.3 | 67.3 ± 6.2 |
| solution | | |
| Normal saline | 22.3 ± 4.7 | 25.0 ± 4 |

TABLE 27
Gel strength (mN) of formulation 2.

Target distance

| Solvent | 5 mm | 10 mm |
|---|---|---|
| Water | 55.3 ± 4.7 | 104.6 ± 2.3 |
| pH 3 | 54 ± 4 | 91.6 ± 13.9 |
| 40% v/v hydroalcoholic | 40.6 ± 2.3 | 54 ± 4 |
| solution | | |
| Normal saline | 20.3 ± 2.3 | 30 ± 4.7 |

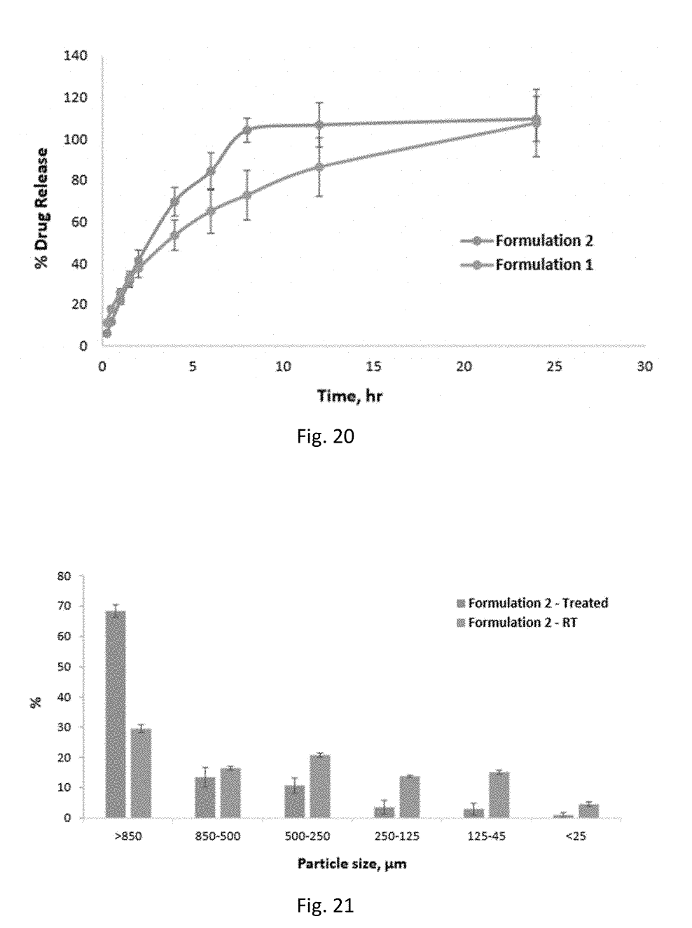

Dissolution Studies: Dissolution study was carried out in USP type II (Paddle) dissolution apparatus at 50 RPM using 900 mL of 0.1 N hydrochloric acid at 37±0.5° C. A tablet was added and the amount of drug released was determined by collecting the solution at specified time intervals and analyzing the solutions using a UV-Vis spectrophotometer (Shimadzu, UV-1700) at a wavelength of 276 nm. Drug release profile was obtained by plotting % drug released from the formulation against time as shown in FIG. 20. A complete controlled drug release was observed with both formulations over 24-hour time period.

Crush resistance: One tablet from formulation 2 was subjected to shear stress using an industry grade high shear grinder (MICROMILL® II, Scienceware Inc.) with ½ hp, 150 watts for 1 min. The crushed tablets were then studied for particle size distribution. The particle size distribution was determined by the weight retained on sieves stacked in a column by sieve number (20, 35, 60, 120, and 325 mesh equivalent to particle sizes of 850, 500, 250, 125 and 45 μm, respectively). Samples were placed into the top sieve and the system loaded into a sieve shaker (Cole Parmer SS-3CP) set to tap for 1 min at 60 taps/min prior to weighing. Crush resistance test showed that tablets were difficult to crush after addition of PEO and heat treatment. The particle size distribution of tablet powder crushed by high shear grinder shows higher percentage of coarser particles after heat treatment of PEO-loaded formulation. Particle size distribution data shown in FIG. 21 also suggests that PEO addition and heat treatment makes tablets difficult to crush into particles of desirable size (typically 150 μm and lower) for abuse by insufflation.

### Example 5: Stability of the Formulations Utilizing the Technologies Disclosed Above

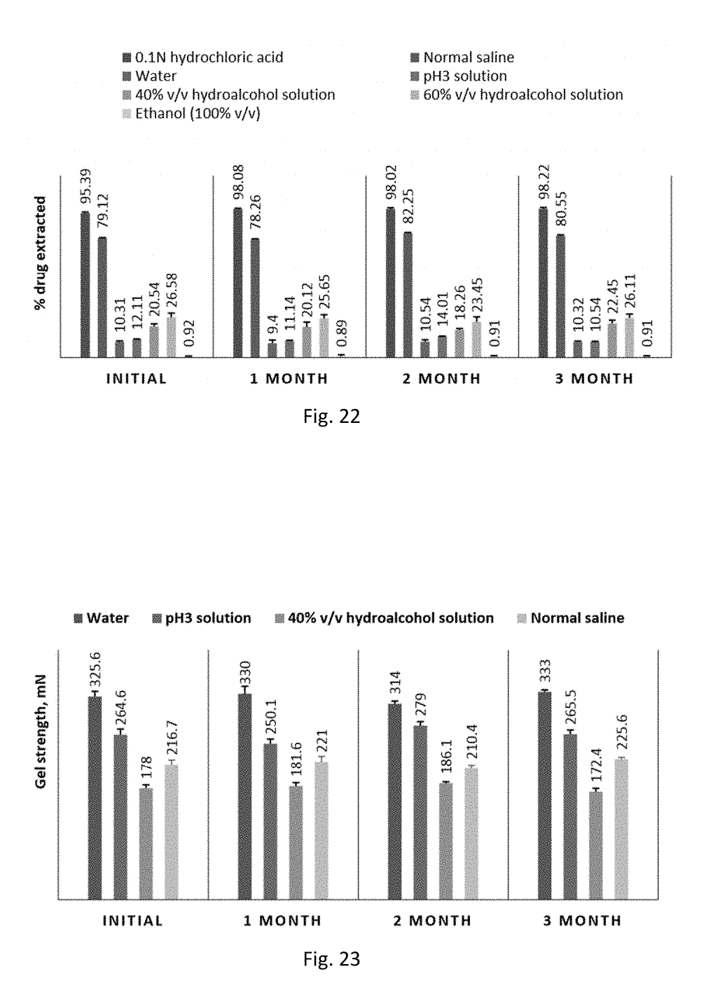

AC-DI-SOL®/Drug Complex: A composition of 320 mg AC-DI-SOL®-Dextromethorphan complex (equivalent to 25 mg Dex) was compressed into a tablet using 180 mg AVICEL®. Tablets were stored at room temperature, and studied for drug extraction stability in different extracting media over three months as shown in FIG. 22.

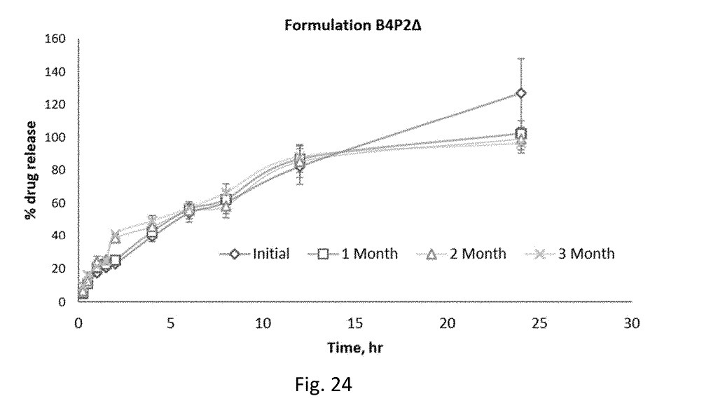

CARBOPOL®/SBC: Tablets composed of 25 mg Dextromethorphan HBr, 150 mg CARBOPOL® 940, 100 mg poly(ethylene oxide) (100,000 Da), 30 mg sodium bicarbonate, and 195 mg AVICEL® were prepared, heated at 80° C. for 30 min, and stored at room temperature. Tablets were studied for their gel strength and drug release stability over three months as shown in FIGS. 23 and 24.

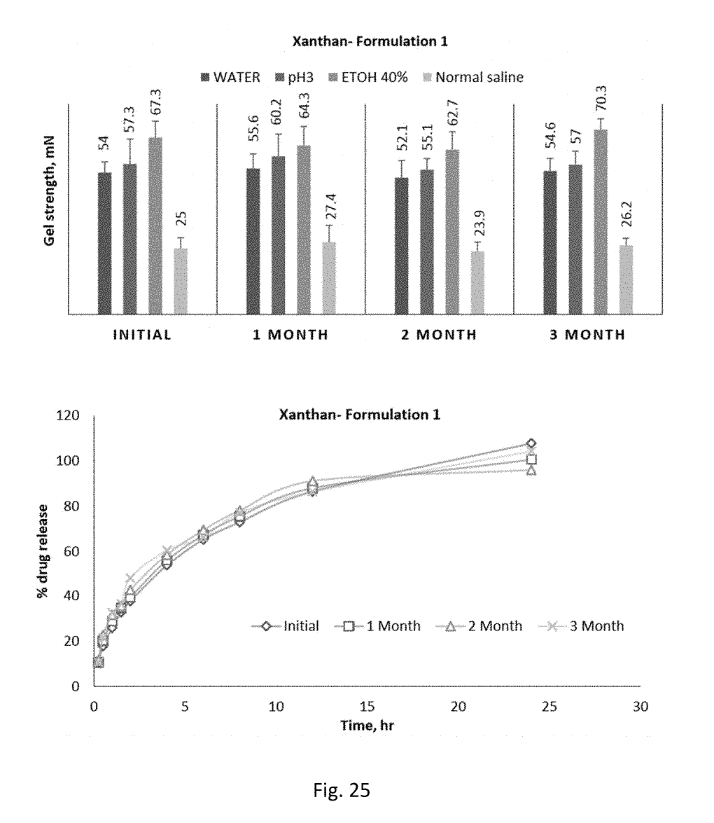

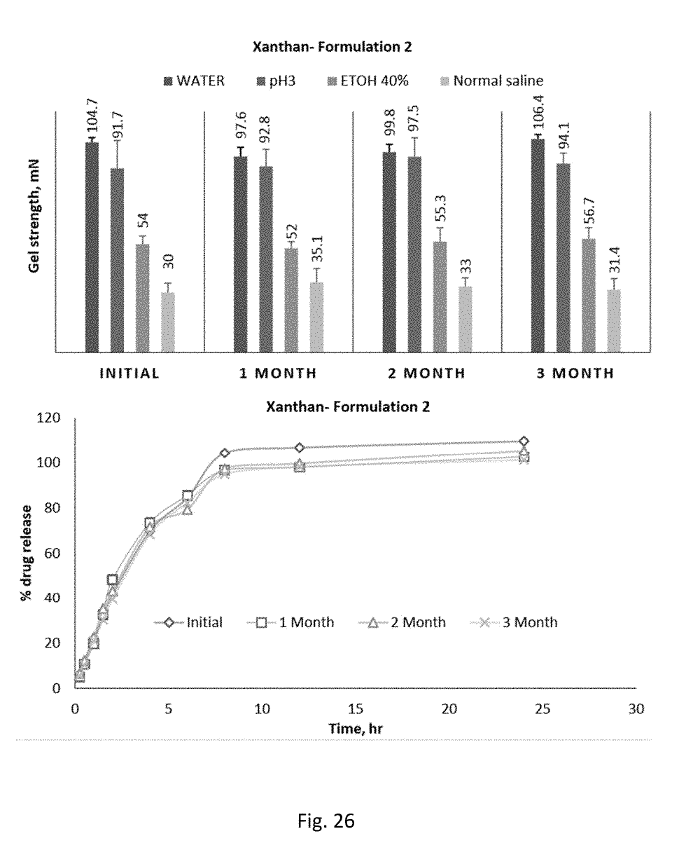

Xanthan Gum: Tablets containing 25 mg Dextromethorphan HBr, 250 mg Xanthan gum, and 125 mg AVICEL® prepared (Formulation 1), and stored at room temperature. Tablets containing 25 mg Dextromethorphan HBr, 250 mg Xanthan gum, 100 mg poly(ethylene oxide) (100,000 Da) and 25 mg AVICEL® prepared, heat treated at 80° C. for 30 min (Formulations 2), and stored at room temperature. Both formulations were studied for their gel strength and drug release stability over three months as shown in FIGS. 25 and 26.

### Example 6: Use of Carbomers to Impart Abuse Deterrence Via Complexation (Binding)

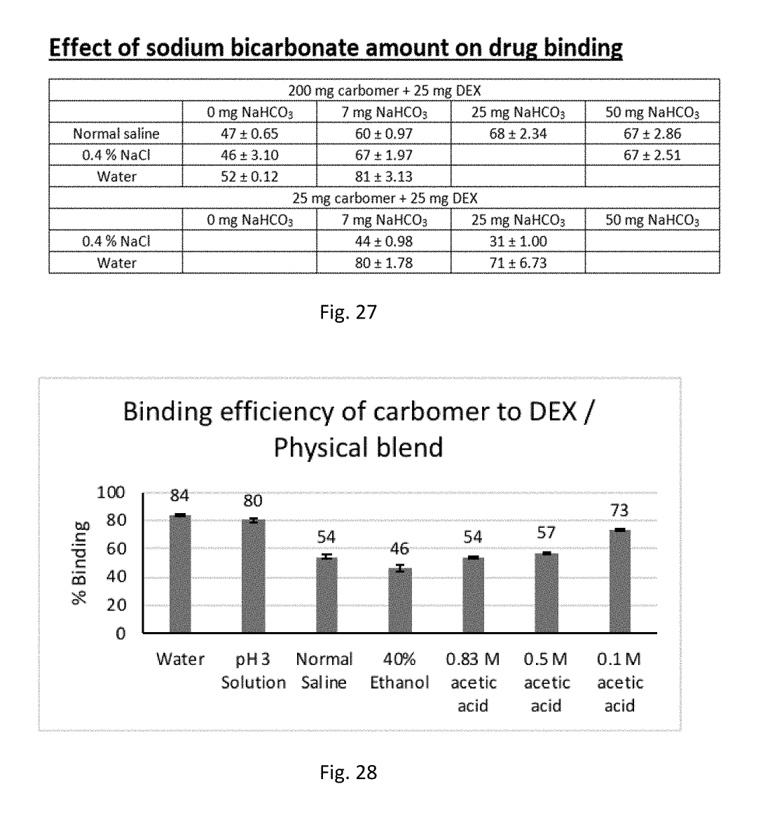

Determination of NaHCO₃ amount required to achieve the highest binding. To mixtures of Carbomer (CARBOPOL® Carbomer Interpolymer Type B; 200 mg, 25 mg), DEX (25 mg), and NaHCO₃ (0, 7, 25, 50 mg), 10 mL solvent (normal saline, 0.4% NaCl, water) was added, and the resulting mixtures were vortexed for 30 sec., and then centrifuged at 2500 rpm (1204 RCF) for 5 min. The supernatants were then filtered, diluted (1 ml to 10 ml) and then measured for absorbance by UV spectrophotometry (@276 nm), using placebo preparations (Carbomer and NaHCO₃ in solvent) as blank. The % assay value was determined and the calculated % binding was determined from the mass balance. Results are shown in FIG. 27. Notes for some preparations: 1) placebo and samples containing 200 mg polymer and 50 mg NaHCO₃ in 0.4% NaCl were prepared in 20 mL, instead of 10 mL; to handle the viscosity for the subsequent analysis steps. This was considered when the dilution step was conducted, so 2 mL were pipetted into 10 mL solution, instead of 1 mL/10 mL; 2) placebo containing 200 mg polymer and 7 mg NaHCO₃ in water was viscous, even after 20 mL water addition; so a placebo containing only 7 mg NaHCO₃ was used instead; and 3) placebo containing 25 mg polymer and 7 mg NaHCO₃ in water was viscous, even after 20 mL water addition; so a placebo containing 25 mg NaHCO₃ was used instead.

Binding efficiency in physical blends. For sample preparation, 200 mg Carbomer (CARBOPOL® Carbomer Interpolymer Type B), 25 mg dextromethorphan HBr, and 7 mg NaHCO₃ were placed in a vial. For placebo preparation, 200 mg Carbomer and 7 mg NaHCO₃ were placed in a vial. To each vial, 10 mL solvent (water, pH3 solution, normal saline, 40% ethanol, and acetic acid solutions (0.83 M, 0.5 M and 0.1 M)) was added, and the resulting mixtures were vortexed for 30 seconds. Notes for placebo preparations: 1) with water, the placebo formed a viscous gel that could not be handled for subsequent analysis steps, so it was prepared using only 7 mg NaHCO₃ as even going with lower Carbomer amount, the mixture was still very difficult to handle; 2) with pH3 solution, the placebo formed a viscous gel that could not be handled for subsequent analysis steps, so it was prepared using 200 mg Carbomer in 20 mL solution instead of 10 mL (this was taken into consideration for subsequent dilution); and 3) with 40% ethanol, the placebo formed a viscous gel that could not be handled for subsequent analysis steps, so it was prepared using 25 mg Carbomer instead of 200 mg. The mixtures were then centrifuged for 5 min. at 2500 rpm (1204 RCF). The supernatants were then filtered (0.2 μm) the supernatant, and suitable dilutions of the supernatants were made (to obtain reasonable UV detection) and the absorbance value of the diluted samples was determined using a UV Spectrophotometer at 276 nm using placebo preparations as blanks. The % assay value was determined and the calculated % binding was determined from the mass balance. Results are shown in FIG. 28.

Binding efficiency in complexes. A low-loaded complex was prepared by stirring together 10 g Carbomer (CARBOPOL® Carbomer Interpolymer Type B), 1.25 g dextromethorphan HBr, and 1.7 g NaHCO₃ in 2500 mL water overnight, using a Stir-Pak (Cole-Parmer) mixer. The mixture was then dried overnight at 65-70° C. using an oven. The unbound drug was washed out by stirring the dried powder with 1 L normal saline for 45 min. at 700 rpm using a stirring plate. The mixture was passed through a mesh (0.85 mm), and the gel masses retained on the mesh were collected for subsequent drying. The gel was dried overnight at 65-70° C. using an oven. The dried powder was then milled using a ball mill to particle size 45 μm<X<125 μm.

A high-loaded complex was prepared by stirring together 8 g Carbomer (CARBOPOL® Carbomer Interpolymer Type B), 6 g dextromethorphan HBr, and 1.7 g NaHCO₃ in 2500 mL water overnight at 550 rpm, using a stirring plate. The mixture was transferred into centrifugation tubes and centrifuged at 4500 rpm (3901 RCF) for 10 min. The sediment was collected and stirred overnight in 2500 mL water to wash out any unbound drug at 550 rpm using a stirring plate. The mixture was then placed into centrifugation tubes and centrifuged at 4500 rpm (3901 RCF) for 10 min. The sediment was collected and dried overnight at 65-70° C. using an oven. The dried powder was then milled using ball mill to particle size 45 μm<X<125 μm.

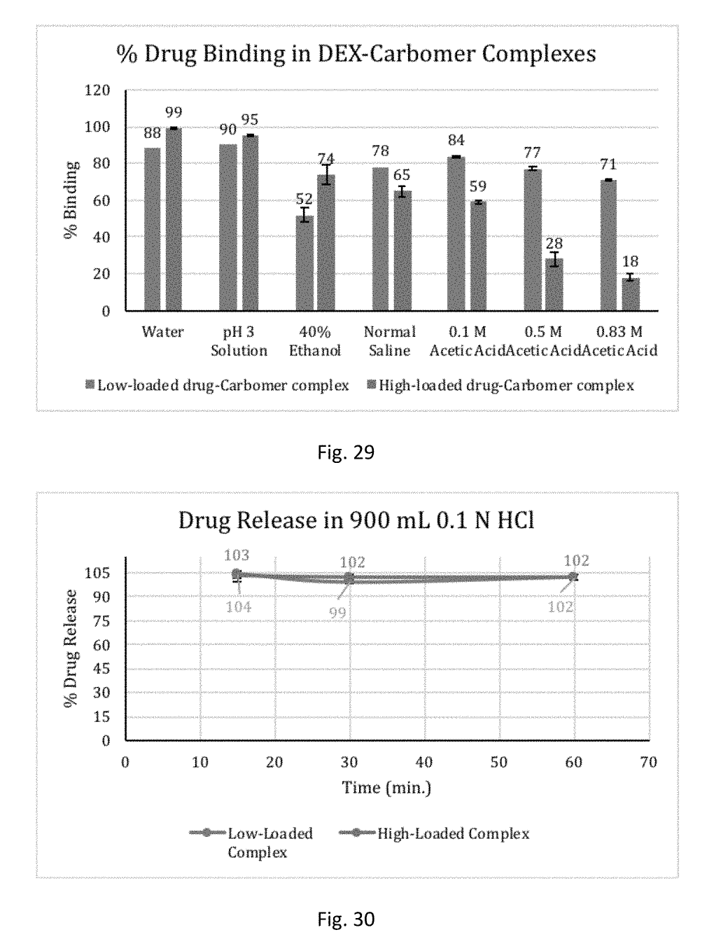

Extraction study from complexes. Ten mL solvent was added to an amount of the complex equivalent to 25 mg DEX; 397.81 mg and 53.75 mg of the low- and high-loaded complexes respectively, and the mixture was vortexed for 30 sec. Note for sample preparation of the high-loaded complex in 40% ethanol: 20 mL solvent was added, instead of 10 mL; to handle the viscosity for the subsequent analysis steps. The mixture was then centrifuged at 2500 rpm (1204 RCF) for 5 min., and the supernatant was collected and filtered. Suitable dilutions were made to obtain reasonable UV detection. Absorbance by UV spectrophotometer (@276 nm) was measured to determine % assay using the solvents as blanks, and the % binding was calculated from the mass balance (FIG. 29).

Drug release from tablets. Tablets were prepared by compressing together the complexes described above with an excipient as follows. Low-loaded complex tablets: 397.81 mg of the complex+102.19 mg AVICEL® PH-101 with placebo tablets: 54.17 mg sodium bicarbonate+318.64 mg Carbomer+102.19 mg AVICEL® PH-101. High-loaded complex tablets: 55 mg of the complex+445 mg AVICEL® PH-101 with placebo tablets: 5.26 mg sodium bicarbonate+24.74 mg Carbomer+445 mg AVICEL® PH-101. Tablets were prepared by direct compression, using single press tableting machine (pressing pressure: 2000 pound).

Phase I dissolution (0.1 N HCl) study. The tablets underwent dissolution testing using Dissolution apparatus (II) paddle at 50 rpm, using 900 mL of 0.1 N HCl as a dissolution medium at 37±0.5° C. Samples (5 mL) were withdrawn from the dissolution vessels after 15 min, 30 min., and 60 min. The withdrawn dissolution medium was immediately replaced after each sampling point using fresh 0.1 N HCl. The percentage of the released drug was measured by a UV spectrophotometer at 276 in reference to a calibration curve established in 0.1N HCl. Results are shown in FIG. 30.

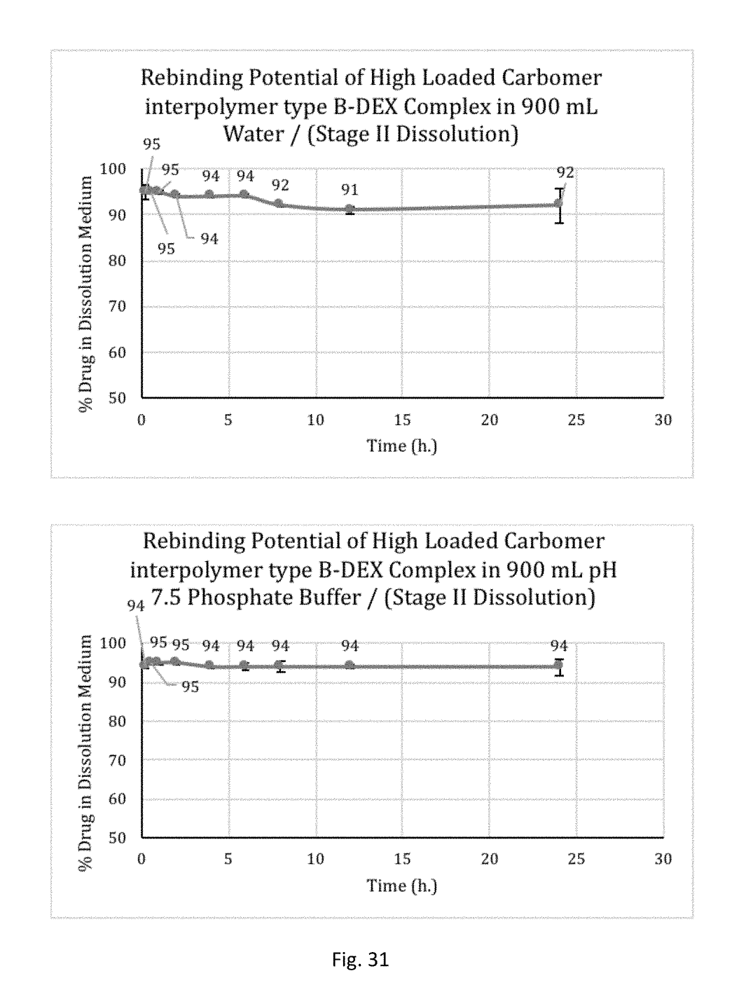

Phase II dissolution study (water and pH 7.5 phosphate buffer). Tablets of the drug-polymer complexes (equivalent to 25 mg DEX) and AVICEL® PH-101 were prepared and run in the dissolution tester (apparatus (II) paddle) using stage I medium (900 mL of 0.1 N HCl) at 50 rpm and 37±0.5° C., along with placebo tablets. The medium containing the released soluble drug from the tablets was dumped, while the insoluble deterring agent (Carbomer) was kept in each dissolution vessel. 900 mL of the new medium (either water or phosphate buffer) were added to the three placebo vessels which already contain protonated deterring agent from stage I dissolution. Drug solution (25 mg drug in 900 mL water or 25 mg drug in 900 mL phosphate buffer) was added to the other three dissolution vessels which again already contain protonated deterring agent from stage I dissolution. The test was run utilizing dissolution apparatus (II) paddle at 50 rpm, 37±0.5° C. and 900 mL of water or pH 7.5 phosphate buffer as a dissolution medium. Samples (5 mL) were withdrawn from the dissolution vessels after 15 min, 30 min., 1 h., 2 h., 4 h., 6 h., 8 h., 12 h., and 24 h. Withdrawn solutions were immediately replaced after each sampling point by fresh dissolution medium. Drug concentration in each dissolution vessel was determined by a UV spectrophotometer at 276 nm, and the percentage of the free drug available was calculated in reference to calibration curves established in water and pH 7.5 phosphate buffer. Results for the high-loaded complex tablets are shown in FIG. 31.

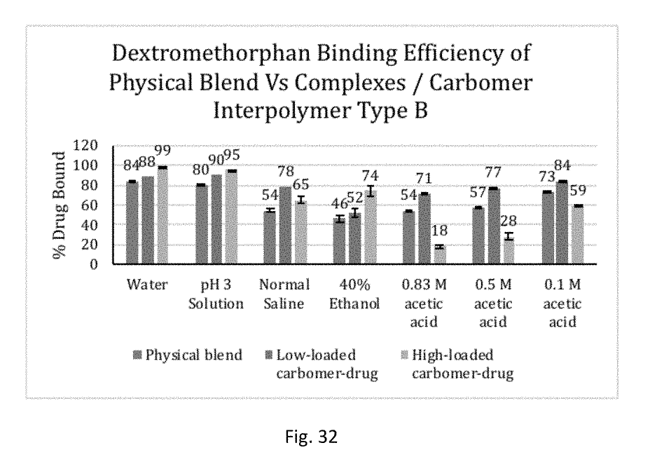

A summary of the above results is provided in FIG. 32 and Tables 28 and 29.

TABLE 28

| | | Low-loaded | High-loaded |
|---|---|---|---|
| | Physical blend | complex | complex |
| Entrapment due to binding | | | |
| Water | 84% | 88% | 99% |
| pH 3 solution | 80% | 90% | 95% |
| 40% ethanol | 46% | 52% | 74% |
| Normal saline | 54% | 78% | 65% |
| 0.83M acetic acid | 54% | 71% | 18% |
| 0.5M acetic acid | 57% | 77% | 28% |
| 0.1M acetic acid | 73% | 84% | 59% |
| Entrapment due to swelling: | | | |
| Water | 7% | | |
| pH 3 solution | 6% | | |
| 40% ethanol | 26% | | |
| Normal saline | 8% | | |
| 0.83M acetic acid | 13% | | |
| 0.5M acetic acid | 12% | | |
| 0.1M acetic acid | 8% | | |
| pH of samples | | | |
| Water | 3.39 | | |
| pH 3 solution | 3.28 | | |
| 40% ethanol | 4.26 | | |
| Normal saline | 2.94 | | |
| 0.83M acetic acid | 2.46 | | |
| 0.5M acetic acid | 2.57 | | |
| 0.1M acetic acid | 2.99 | | |

TABLE 29

| Sediment height | |
|---|---|
| Water | 1.10 cm |
| pH 3 solution | 0.90 cm |
| 40% ethanol | 0.85 cm |
| Normal saline | 0.60 cm |
| 0.83M acetic acid | 0.90 cm |
| 0.5M acetic acid | 0.80 cm |
| 0.1M acetic acid | 0.85 cm |
| Drug released (Stage I) in 0.1N | |
| HCl after 15 min. | |
| Low-loaded complex | 104% |
| High-loaded complex | 103% |
| Drug released (Stage II) in | |
| water after 24 hours | |
| Low-loaded complex | NA |
| High-loaded complex | 92% |
| Drug released (Stage II) in | |
| pH 7.5 phosphate buffer | |
| after 24 hours | |
| Low-loaded complex | NA |
| High-loaded complex | 94% |

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An abuse-resistant therapeutic pharmaceutical formulation comprising: a cationic drug and at least one anionic polymer; wherein (i) the cationic drug is physically blended with but not ionically bound to the at least one anionic polymer, or (ii) more than 40% of the cationic drug is ionically bound (complexed) to the at least one anionic polymer; wherein the formulation prevents the cationic drug from being extracted from the formulation in a solvent selected from the group consisting of water, hydroalcohol solutions, pH 3 solutions, acetic acid solutions, and saline, at solution temperatures of 20-90° C.

2. The pharmaceutical formulation of claim 1, wherein the at least one anionic polymer comprises a poly(acrylic acid) homopolymer, copolymer, terpolymer, or interpolymer.

3. The pharmaceutical formulation of claim 2, wherein the poly(acrylic acid) homopolymer, copolymer, terpolymer, or interpolymer is crosslinked with an allyl ether of pentaerythritol.

4. The pharmaceutical formulation of claim 2, wherein the poly(acrylic acid) homopolymer, copolymer, terpolymer, or interpolymer is crosslinked with an allyl ether of sucrose.

5. The pharmaceutical formulation of claim 2, wherein the poly(acrylic acid) homopolymer, copolymer, terpolymer, or interpolymer is crosslinked with an allyl ether of propylene.

6. The pharmaceutical formulation of claim 2, wherein the poly(acrylic acid) homopolymer, copolymer, terpolymer, or interpolymer is crosslinked with a vinyl crosslinker.

7. The pharmaceutical formulation of claim 2, wherein the cationic drug is physically blended with but not ionically bound to the poly(acrylic acid) homopolymer, copolymer, terpolymer, or interpolymer and the formulation further comprises an alkalinizing agent, wherein the weight ratio of the alkalizing agent to the poly(acrylic acid) homopolymer, copolymer, terpolymer, or interpolymer is between about 2% to 20%.

8. The pharmaceutical formulation of claim 7, wherein the weight ratio of the alkalizing agent to the poly(acrylic acid) homopolymer, copolymer, terpolymer, or interpolymer is between about 2% to 10%.

9. The pharmaceutical formulation of claim 7, wherein the weight ratio of the alkalizing agent to the poly(acrylic acid) homopolymer, copolymer or interpolymer is about 3.5%.

10. The pharmaceutical formulation of claim 7, wherein the alkalinizing agent is a bicarbonate salt.

11. The pharmaceutical formulation of claim 1, wherein the cationic drug is ionically bound (complexed) to the at least one anionic polymer.

12. The pharmaceutical formulation of claim 11, wherein the at least one anionic polymer comprises a poly(acrylic acid) homopolymer, copolymer, terpolymer, or interpolymer.

13. The pharmaceutical formulation of claim 12, wherein the cationic drug-poly(acrylic acid) homopolymer, copolymer, terpolymer, or interpolymer complex is made by reacting the cationic drug and the poly(acrylic acid) homopolymer, copolymer, terpolymer, or interpolymer in an aqueous solution at a pH range of greater than the pKa−1 of the poly(acrylic acid) homopolymer, copolymer, terpolymer, or interpolymer and lower than the pKa+1 of the cationic drug.

14. The pharmaceutical formulation of claim 13, wherein the aqueous solution comprises an alkalinizing agent that causes the pH range of the aqueous solution to be greater than the pKa−1 of the poly(acrylic acid) homopolymer, copolymer, terpolymer, or interpolymer and lower than the pKa+1 of the cationic drug.

15. The pharmaceutical formulation of claim 14, wherein the alkalinizing agent is a bicarbonate salt.
